(19) United States
(12) Patent Application Publication
(10) Pub. No.: US 2019/0361025 A1
(43) Pub. Date: Nov. 28, 2019

(54) LIQUID BIOPSIES FOR DETECTION OF PROSTATE CANCER

(71) Applicant: Sanford Burnham Prebys Medical Discovery Institute, La Jolla, CA (US)

(72) Inventor: Ranjan J. PERERA, La Jolla, CA (US)

(21) Appl. No.: 16/484,791

(22) PCT Filed: Feb. 9, 2018
(86) PCT No.: PCT/US2018/017687

§ 371 Date: Aug. 8, 2019

Related U.S. Application Data
(60) Provisional application No. 62/457,424, filed on Feb. 10, 2017.

(51) Int. Cl. G01N 33/574; C12Q 1/6886

(52) U.S. Cl. CPC G01N 33/57434; C12Q 1/6886; G01N 2800/52; C12Q 2600/178; C12Q 2600/106; C12Q 2600/158

(57) ABSTRACT

Methods and compositions are provided for detecting, diagnosing and evaluating presence of prostate cancer in a biological sample.

### CROSS REFERENCE TO RELATED APPLICATION(S)

This application claims the benefit of priority under 35 U.S.C. § 119(e) of U.S. Ser. No. 62/457,424, filed Feb. 10, 2017, the entire contents of which is incorporated herein by reference in its entirety.

### STATEMENT OF GOVERNMENT SUPPORT

This invention was made in part with government support under Grant Nos. 5P30CA030199 and U24 DK097209, all of which were awarded by the National Institutes of Health. The United States government has certain rights in this invention.

### INCORPORATION OF SEQUENCE LISTING

The material in the accompanying sequence listing is hereby incorporated by reference into this application. The accompanying sequence listing text file, name BURN1740_1WO_Sequence_Listing.txt, was created on Feb. 7, 2018, and is 6 kb. The file can be assessed using Microsoft Word on a computer that uses Windows OS.

### BACKGROUND

### Field of Invention

The present invention relates generally to methods for cancer detection, and more particularly to methods and compositions for diagnosing prostate cancer.

### Background Information

More than 180,000 men were diagnosed with prostate cancer (PCa) in the U.S. in 2016; 26,000 of these patients will die of the disease. PCa is the second most frequently diagnosed cancer and the fifth leading cause of cancer deaths in men worldwide. Although radiotherapy and surgery for localized PCa are effective, the prognosis for patients with advanced disease is poor. A test to detect PCa with high sensitivity and specificity at an early stage is imperative. Moreover, there is an urgent need for novel therapeutic approaches to manage this insidious and prevalent disease.

Serum prostate specific antigen (PSA) levels have been used for PCa diagnosis and screening for over thirty years, and digital rectal examination (DRE) for even longer. However, PSA has poor sensitivity and specificity and does not distinguish indolent from aggressive cancers. Prostate cancer antigen 3 (PCA3), a prostate-specific non-coding RNA, was approved by the FDA in 2012 as the first PCa molecular diagnostic test for a specific clinical indication (need for repeat prostate biopsies in men aged >50 years with suspected PCa based on PSA levels and/or DRE and/or one or more previous negative biopsies). However, the value of the PCA3 test is limited by significant individual variability, better performance in the repeat biopsy setting, and conflicting data on the relationship between score and cancer grade using the most common threshold of 35. Hence, there is a dire need for novel molecular diagnostic tools to more accurately detect and predict the behavior of localized PCa.

The kidneys produce urine to eliminate soluble waste from the bloodstream. Urine is an abundant biofluid for molecular or cellular analyses and is useful in the diagnosis and management of bladder, ovarian, and kidney diseases. Urine contains over 2500 metabolites that provide a window for viewing cellular biochemical reactions and intermediary metabolism. The metabolite signature in urine will reflect the impact of gene regulation, enzyme activities, and alterations in metabolic reactions from the different cell types found along the urogenital tract.

Cancer cells exhibit perturbed metabolism that enables their proliferative behavior. Therefore, metabolomic profiling has been a fruitful approach for the identification of early cancer biomarkers. Furthermore, certain metabolic states are associated with prognosis in advanced cancers. Several metabolomics studies have revealed PCa-specific metabolic phenotypes in serum, tissue, and urine. Indeed, an intermediate metabolite of glycine synthesis and degradation, sarcosine, has been described as a putative PCa biomarker in urine. However, the utility of sarcosine as a biomarker is controversial and clinical validation has been elusive.

Metabolomics data have been integrated with comprehensive gene expression analysis to interrogate complex gene and metabolic networks. Integrating multiple aspects of biological complexity using different unsupervised approaches serves to pinpoint the most important and reproducible pathways driving the biological processes and hence reveal robust biomarkers or promising drug targets.

There exists a need for more sensitive and specific diagnostic and prognostic biomarkers for prostate cancer.

### SUMMARY OF THE INVENTION

The present invention is based on the discovery that an integrated analysis of gene expression and metabolite signatures provide greater insight into prostate cancer diagnosis and prognosis. Methods and compositions are provided to predict, evaluate, diagnose, and monitor cancer, particularly prostate cancer, by measuring certain biomarkers in biological sample, including but not limited to serum, plasma, feces, or urine.

Accordingly, in one embodiment, the invention provides a method of performing an assay. The method includes: a) detecting an expression level of a gene in a sample from a subject having or suspected of having prostate cancer, wherein the gene is selected from one or more genes recited in Table 2, Table 3 and/or Table 6; and/or b) detecting a level of a metabolite in the sample, wherein the metabolite comprises one or more amino acids, organic acids or acylcarnitine derivatives as set forth in Table 7 and/or Table 11.

In another embodiment, the invention provides a method of determining and administering a treatment for prostate cancer in a subject having or suspected of having prostate cancer. The method includes: a) determining a treatment for prostate cancer in a subject having or suspected of having prostate cancer by performing the assay of the present invention; and b) administering a treatment to the subject. In embodiments, the treatment includes administering a chemotherapeutic agent and/or treatment therapy.

In yet another embodiment, the invention provides a method of diagnosing cancer in a sample from a subject having or at risk of having prostate cancer. The method includes: a) detecting an expression level of a gene in a sample from a subject having or suspected of having prostate cancer, wherein the gene is selected from one or more genes recited in Table 2, Table 3 and/or Table 6; and optionally, detecting a level of a metabolite in the sample, wherein the metabolite comprises one or more amino acids, organic acids or acylcarnitine derivatives as set forth in Table 7 and/or Table 11; and b) diagnosing cancer in the subject, wherein the expression level, or level of the metabolite in the sample, being up-regulated or down-regulated as compared to a corresponding normal sample is indicative of prostate cancer, thereby diagnosing cancer in the subject.

In still another embodiment, the invention provides a probe set for detecting or assessing prostate cancer. The probe set includes a plurality of probes, wherein each probe is capable of detecting an expression level of a gene selected from those recited in Table 2, Table 3 and/or Table 6.

The invention further provides an array which includes a plurality of probes for detecting the expression level of a gene selected from those recited in Table 2, Table 3 and/or Table 6. In various embodiments, the probes are oligonucleotides, polypeptides or antibodies.

In another embodiment, the invention provides a kit that includes the probe set, or array of the present invention, and/or reagents for detecting a level of a metabolite in a sample, wherein the metabolite comprises one or more amino acids, organic acids or acylcarnitine derivatives as set forth in Table 7 and/or Table 11.

In one embodiment, the invention provides a method of inhibiting the growth of prostate cancer cells comprising administering to the cell a GOT1 inhibitor, thereby reducing the growth of the prostate cancer cells. Such inhibitors may include but are not limited to small molecule, a peptide, or a nucleic acid molecule. For example, the inhibitor can be an siRNA molecule.

### BRIEF DESCRIPTION OF THE DRAWINGS

FIG. 1A is a graphical plot representing data related to one embodiment of the invention.

FIG. 1B is a graphical plot representing data related to one embodiment of the invention.

FIG. 1C is a graphical plot representing data related to one embodiment of the invention.

FIG. 1D is a graphical plot representing data related to one embodiment of the invention.

FIG. 2A is a graphical plot representing data related to one embodiment of the invention.

FIG. 2B is a graphical representation of a heat map related to one embodiment of the invention.

FIG. 2C is a graphical representation of a heat map related to one embodiment of the invention.

FIG. 2D is a series of graphical plots representing data related to one embodiment of the invention.

FIG. 3A is a graphical plot representing data related to one embodiment of the invention.

FIG. 3B is a graphical illustration showing integrative pathway analysis related to one embodiment of the invention.

FIG. 4A is a graphical plot representing data related to one embodiment of the invention.

FIG. 4B is a series of graphical plots representing data related to one embodiment of the invention.

FIG. 4C is a series of graphical plots representing data related to one embodiment of the invention.

FIG. 4D is a series of graphical plots representing data related to one embodiment of the invention.

FIG. 4E is a series of graphical plots representing data related to one embodiment of the invention.

FIG. 5A is a series of graphical plots representing data related to one embodiment of the invention.

FIG. 5B is a series of graphical plots representing data related to one embodiment of the invention.

FIG. 5C is a series of graphical plots representing data related to one embodiment of the invention.

FIG. 6A is a pictorial representation of a clustering tree related to one embodiment of the invention.

FIG. 6B is a pictorial representation of principal component analysis related to one embodiment of the invention.

FIG. 7A is a series of graphical plots representing data related to one embodiment of the invention.

FIG. 7B is a series of graphical plots representing data related to one embodiment of the invention.

FIG. 7C is a series of graphical plots representing data related to one embodiment of the invention.

FIG. 8 is a graphical schematic illustrating TCA cycle and glutamine metabolism.

FIG. 9A is a series of graphical plots representing data related to one embodiment of the invention.

FIG. 9B is a series of graphical plots representing data related to one embodiment of the invention.

FIG. 10A is a series of graphical plots representing data related to one embodiment of the invention.

FIG. 10B is a series of graphical plots representing data related to one embodiment of the invention.

FIG. 11A is a table showing data related to one embodiment of the invention.

FIG. 11B is a table showing data related to one embodiment of the invention.

FIG. 11C is a table showing data related to one embodiment of the invention.

FIG. 12A is a table showing data related to one embodiment of the invention. The table depicts metabolites showing significantly different levels between clinical groups (BPH, prostatitis, or PCa urine samples versus normal).

FIG. 12B is a table showing data related to one embodiment of the invention. The table depicts metabolites showing significantly different levels between clinical groups (BPH, prostatitis, or PCa urine samples versus normal).

FIG. 12C is a table showing data related to one embodiment of the invention. The table depicts metabolites showing significantly different levels between clinical groups (BPH, prostatitis, or PCa urine samples versus normal).

### DESCRIPTION OF THE INVENTION

The present invention relates to an integrated approach to assessing prostate cancer including analysis of gene expression and metabolite signatures. Described herein are methods to detect, evaluate, diagnose, prognose and monitor prostate cancer.

The present disclosure describes metabolite profiling (global and targeted) and high-throughput RNA sequencing that was performed of urine from patients with benign prostatic hyperplasia (BPH), prostatitis, and Prostate cancer (PCa). The aims were to (a) discover cancer-specific changes in the urine with sensitive and specific PCa biomarkers either alone or in combination and (b) identify novel drug targets for PCa. Importantly, the approach used single void urine samples (i.e., without prostatic massage) as proof-of-principle of how urine can be used in biomarker and target discovery. Integrated analysis of metabolomic and transcriptomic data from these liquid biopsies revealed a glutamate (Glu) metabolism and tricarboxylic acid (TCA) cycle node that was specific to prostate-derived cancer cells and cancer-specific metabolic changes in urine. Functional validation in vitro provided mechanistic support for a pivotal role for GOT1-dependent Glu metabolism in redox balance and cancer progression.

Before the present compositions and methods are further described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The presently disclosed subject matter provides a method and composition utilizing biomarkers, specifically expression products of various genes, in combination with metabolites including amino acids, organic acids and/or acylcarnitine derivatives, that are useful for the detection, desirably early detection, of prostate cancer. The biomarkers provided herein address certain limitations of early detection of tumors by other methods of screening alone.

A “biomarker” in the context of the present invention is a molecular indicator of a specific biological property; a biochemical feature or facet that can be used to measure the progress of disease or the effects of treatment. “Biomarker” encompasses, without limitation, expression products of genes, including ma and proteins, as well as their polymorphisms, mutations, variants, modifications, subunits, fragments, complexes, unique epitopes, and degradation products thereof. “Biomarker” further encompasses, without limitation, amino acids, organic acids or acylcarnitine derivatives as well as modifications, subunits, fragments, complexes, unique epitopes, and degradation products thereof.

The term “polypeptide” or “protein” is used in its broadest sense to refer to a polymer of subunit amino acids, amino acid analogs, or peptidomimetics, including proteins and peptoids. The polypeptides may be naturally occurring full length proteins or fragments thereof, processed forms of naturally occurring polypeptides (such as by enzymatic digestion), chemically synthesized polypeptides, or recombinantly expressed polypeptides. The polypeptides may comprise D- and/or L-amino acids, as well as any other synthetic amino acid subunit, and may contain any other type of suitable modification, including but not limited to peptidomimetic bonds and reduced peptide bonds.

Accordingly, in one embodiment, the invention provides a method of performing an assay. The method includes: a) detecting an expression level of a gene in a sample from a subject having or suspected of having prostate cancer, wherein the gene is selected from one or more genes recited in Table 2, Table 3 or Table 6; and optionally, b) detecting a level of a metabolite in the sample, wherein the metabolite comprises one or more amino acids, organic acids or acylcarnitine derivatives as set forth in Table 7.

In embodiments, the one or more genes is selected from those determined to be upregulated in prostate cancer as set forth in Table 2, and those determined to be downregulated in prostate cancer as set forth in Table 3. In one embodiment, the one or more genes is selected from EPCAM, GRHPR, HDAC6, PHB, RPS11, GOT1, ELK4, SMARCB1, BRD3, TFG, NACA, NPM1, RPL22, and any combination thereof. In one embodiment, the one or more genes consists of EPCAM, GRHPR, HDAC6, PHB, RPS11, GOT1, ELK4, SMARCB1, BRD3, TFG, NACA, NPM1, and RPL22. In embodiments, the one or more genes is selected from those determined to be upregulated in prostate cancer, such as one or more of BRD3, ELK4, EPCAM, FH, GRHPR, HDAC6, NACA, NPM1, PHB, RPL22, RPS11, SMARCB1, TFG, and combinations thereof. In one embodiment, the one or more genes consists of BRD3, ELK4, EPCAM, FH, GRHPR, HDAC6, NACA, NPM1, PHB, RPL22, RPS11, SMARCB1 and TFG.

In embodiments the one or more amino acids or derivatives thereof is selected from those set forth in Table 7. For example, the one or more amino acids or derivatives thereof is selected from 1-Methylhistidine, Alanine, Aspartate, Citrulline, Glutamate, Glutamine, Histidine, Isoleucine, Lysine, Methionine, Ornithine, Proline, Threonine, Tyrosine and combinations thereof. In one embodiment, the one or more amino acids or derivatives thereof is selected from 1-Methylhistidine, Alanine, Aspartate, Citrulline, Glutamate, Glutamine, Histidine, Isoleucine, Lysine, Methionine, Ornithine, Proline, Threonine, Tyrosine, L-Aspartate, L-Proline, N-Acetylglycine, 5-oxo-L-Proline, 3-sulfino-L-Alanine, beta-Alanine, N-methyl-D-Aspartate, N-Acetyl-L-Leucine, creatine, and combinations thereof. In one embodiment, the one or more amino acids or derivatives thereof consists of 1-Methylhistidine, Aspartate, Glutamate, Isoleucine, Ornithine and Proline. In one embodiment, the one or more amino acids or derivatives thereof is Aspartate, Glutamate or combination thereof. In one embodiment, the one or more amino acids or derivatives thereof is L-Aspartate, L-Proline, N-Acetylglycine, 5-oxo-L-Proline, 3-sulfino-L-Alanine, beta-Alanine, N-methyl-D-Aspartate, N-Acetyl-L-Leucine, creatine, and combinations thereof. In one embodiment, the one or more amino acids or derivatives thereof consists of L-Aspartate, L-Proline, N-Acetylglycine, 5-oxo-L-Proline, 3-sulfino-L-Alanine, beta-Alanine, N-methyl-D-Aspartate, N-Acetyl-L-Leucine, and creatine.

In embodiments the one or more organic acids is selected from those set forth in Table 7. For example, the one or more organic acids is selected from Pyruvate, Succinate, Malate and α-ketoglutarate. In embodiments the one or more organic acids consists of Pyruvate, Succinate, Malate and α-ketoglutarate. In embodiments the one or more organic acids is Pyruvate, Succinate, Malate and α-ketoglutarate, 3-hydroxy-3-Methylglutarate, 4-Guanidinobutanoate, D-Glucuronolactone and combinations thereof. In embodiments the one or more organic acids consists of Pyruvate, Succinate, Malate and α-ketoglutarate. In embodiments the one or more organic acids is Pyruvate, Succinate, Malate and α-ketoglutarate, 4-Guanidinobutanoate, 3-hydroxy-3-Methylglutarate, and D-Glucuronolactone. In embodiments the one or more organic acids is 3-hydroxy-3-Methylglutarate, D-Glucuronolactone, Succinate and combinations thereof. In embodiments the one or more organic acids consists of 3-hydroxy-3-Methylglutarate, 4-Guanidinobutanoate, D-Glucuronolactone and Succinate.

In embodiments the one or more acylcarnitine derivatives is selected from those set forth in Table 7. For example, the one or more acylcarnitine derivatives is selected from C3, C3-DC, C4 isobutyryl, C4-methylmanlonyl, C4-OH butyryl, C4-OH isobutyryl, C5 2-methylbutyryl, C5 isovaleryl, C6, C6-OH and C10 acylcarnitine derivatives.

In one embodiment, the metabolite is upregulated and selected from one or more of L-Aspartate, L-Proline, N-Acetylglycine, 5-oxo-L-Proline, 3-sulfino-L-Alanine, beta-Alanine, N-methyl-D-Aspartate, N-Acetyl-L-Leucine, creatine, 3-hydroxy-3-Methylglutarate, 4-Guanidinobutanoate, D-Glucuronolactone, Succinate and combinations thereof. In one embodiment, the metabolite is upregulated and consists of L-Aspartate, L-Proline, N-Acetylglycine, 5-oxo-L-Proline, 3-sulfino-L-Alanine, beta-Alanine, N-methyl-D-Aspartate, N-Acetyl-L-Leucine, creatine, 3-hydroxy-3-Methylglutarate, D-Glucuronolactone, and Succinate.

In one embodiment, the metabolite is selected from one or more of L-Aspartate, L-Proline, N-Acetylglycine, 5-oxo-L-Proline, 3-sulfino-L-Alanine, beta-Alanine, N-methyl-D-Aspartate, N-Acetyl-L-Leucine, creatine, 3-hydroxy-3-Methylglutarate, 4-Guanidinobutanoate, D-Glucuronolactone, Succinate and combinations thereof; and the one or more genes is selected from BRD3, ELK4, EPCAM, FH, GRHPR, HDAC6, NACA, NPM1, PHB, RPL22, RPS11, SMARCB1, TFG, and combinations thereof. In one embodiment, the metabolite consists of L-Aspartate, L-Proline, N-Acetylglycine, 5-oxo-L-Proline, 3-sulfino-L-Alanine, beta-Alanine, N-methyl-D-Aspartate, N-Acetyl-L-Leucine, creatine, 3-hydroxy-3-Methylglutarate, 4-Guanidinobutanoate, D-Glucuronolactone, and Succinate; and the one or more genes consists of BRD3, ELK4, EPCAM, FH, GRHPR, HDAC6, NACA, NPM1, PHB, RPL22, RPS11, SMARCB1, and TFG.

The level of a biomarker of the present invention can be determined using the presently disclosed assay method. In some embodiments, the biomarkers can comprise one or more gene expression products and at least one or more metabolites, such as one or more amino acids, organic acids or acylcarnitine derivatives. However, the presently disclosed subject matter is not limited to biomarkers as described above. Any marker that correlates with prostate cancer or the progression of prostate cancer can be included in the biomarker panel provided herein, and is within the scope of the presently disclosed subject matter. Any suitable method can be utilized to identify additional prostate cancer biomarkers suitable for use in the presently disclosed methods. For example, biomarkers that are known or identified as being up or down-regulated in prostate cancer using methods known to those of ordinary skill in the art can be employed. Additional biomarkers can include one or more of polypeptides, small molecule metabolites, lipids and nucleotide sequences. Markers for inclusion on a panel can be selected by screening for their predictive value using any suitable method, including but not limited to, those described.

As is apparent from the foregoing embodiments, the presently disclosed method is useful for screening patients for prostate cancer, for the early detection of prostate cancer, and for managing the treatment of patients with potential prostate cancer or with known prostate cancer. For example, in some embodiments, the panel of biomarkers can be useful for screening patients prior to other known methods for detecting tumors, to define patients at high risk or higher risk for prostate cancer. Further, the presently disclosed method may be utilized in combination with other screening methods, such as histological analysis.

In one embodiment, the presence of any amount of biomarker in a sample from a subject at risk of prostate cancer can indicate a likelihood of prostate cancer in the subject. In another embodiment, if biomarkers are present in a sample from a subject at risk of prostate cancer, at levels which are higher than that of a control sample (a sample from a subject who does not have prostate cancer) than the subject at risk of prostate cancer has a likelihood of prostate cancer. Subjects with a likelihood of prostate cancer can then be tested for the actual presence of prostate cancer using standard diagnostic techniques known to the skilled artisan, including biopsy, histological analysis and the like. In various embodiments, the method results in an accurate diagnosis in at least 70% of cases; more preferably of at least 75%, 80%, 85%, 90%, 95%, 96%, 97%, 98%, 99%, or more of the cases.

Any suitable method can be employed for determining the level of a biomarker, as would be apparent to one skilled in the art upon a review of the present disclosure. For example, gene expression levels may be detected by measuring protein levels. A variety of detection techniques are suitable for detection of proteins. For example, methods for detecting proteins can include gas chromatography (GC), liquid chromatography/mass spectroscopy (LC-MS), gas chromatography/mass spectroscopy (GC-MS), nuclear magnetic resonance (NMR), magnetic resonance imaging (MM), Fourier Transform InfraRed (FT-IR), and inductively coupled plasma mass spectrometry (ICP-MS). It is further understood that mass spectrometry techniques include, but are not limited to, the use of magnetic-sector and double focusing instruments, transmission quadrapole instruments, quadrupole ion-trap instruments, time-of-flight instruments (TOF), Fourier transform ion cyclotron resonance instruments (FT-MS), and matrix-assisted laser desorption/ionization time-of-flight mass spectrometry (MALDI-TOF MS).

In some embodiments, protein biomarkers can be detected using technologies well known to those of skill in the art such as gel electrophoresis, immunohistochemistry, and antibody binding. Methods for generating antibodies against a polypeptide of interest are well known to those of ordinary skill in the art. An antibody against a protein biomarker of the presently disclosed subject matter can be any monoclonal or polyclonal antibody, so long as it suitably recognizes the protein biomarker. In some embodiments, antibodies are produced using the protein biomarker as the immunogen according to any conventional antibody or antiserum preparation process. The presently disclosed subject matter provides for the use of both monoclonal and polyclonal antibodies. In addition, a protein used herein as the immunogen is not limited to any particular type of immunogen. For example, fragments of the protein biomarkers of the presently disclosed subject matter can be used as immunogens. The fragments can be obtained by any method including, but not limited to, expressing a fragment of the gene encoding the protein, enzymatic processing of the protein, chemical synthesis, and the like.

Antibodies of the presently disclosed subject matter can be useful for detecting the protein biomarkers. For example, antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, and the like), complement fixation assays, immunofluorescence assays, protein A assays, and immunoelectrophoresis assays, and the like. Upon review of the present disclosure, those skilled in the art will be familiar with numerous specific immunoassay formats and variations thereof that can be useful for carrying out the methods of the presently disclosed subject matter.

In any embodiment of the invention, detection techniques may utilize a detectable tag, such as a detectable moiety. A tag may be linked to a polypeptide through covalent bonding, including, but not limited to, disulfide bonding, hydrogen bonding, electrostatic bonding, recombinant fusion and conformational bonding. Alternatively, a tag may be linked to a polypeptide by means of one or more linking compounds. Techniques for conjugating tags to polypeptides are well known to the skilled artisan. Detectable tags can be used diagnostically to, for example, assess the presence of antibodies, or antibodies to a protein in a sample; and thereby detect the presence of prostate cancer, or monitor the development or progression of prostate cancer as part of a clinical testing procedure. Any suitable detection tag can be used, including but not limited to enzymes, prosthetic groups, fluorescent materials, luminescent materials, bioluminescent materials, radioactive materials, positron emitting metals, and nonradioactive paramagnetic metal ions. The tag used will depend on the specific detection/analysis/diagnosis techniques and/or methods used such as immunohistochemical staining of (tissue) samples, flow cytometric detection, scanning laser cytometric detection, fluorescent immunoassays, enzyme-linked immunosorbent assays (ELISAs), radioimmunoassays (RIAs), bioassays (e.g., neutralization assays), Western blotting applications, and the like. For immunohistochemical staining of tissue samples preferred tags are enzymes that catalyze production and local deposition of a detectable product. Enzymes typically conjugated to polypeptides to permit their immunohistochemical visualization are well known and include, but are not limited to, acetylcholinesterase, alkaline phosphatase, beta-galactosidase, glucose oxidase, horseradish peroxidase, and urease. Typical substrates for production and deposition of visually detectable products are also well known to the skilled person in the art. The polypeptides can be labeled using colloidal gold or they can be labeled with radioisotopes.

Gene expression levels may be determined in a disclosed method using any technique known in the art. Exemplary techniques include, for example, methods based on hybridization analysis of polynucleotides (e.g., genomic nucleic acid sequences and/or transcripts (e.g., mRNA)), methods based on sequencing of polynucleotides, methods based on detecting proteins (e.g., immunohistochemistry and proteomics-based methods).

The assays described herein can be adapted to be performed by lay users without a laboratory. The users may be health care professionals in point-of-care facilities or lay consumers in field conditions. The devices may have multiple embodiments including single-use devices, simple reusable devices and computerized biomonitors. The single-use devices, similar to over-the-counter lateral flow assays for pregnancy, enable subjective multi-biomarker assays to be performed. Simple reusable devices also enable objective biomarker assays that provide a refined or enhanced indication of solid state cancer mass, and may also enable remote data processing.

Gene expression levels also can be determined by quantification of a microRNA or gene transcript (e.g., mRNA). Commonly used methods known in the art for the quantification of mRNA expression in a sample include, without limitation, northern blotting and in situ hybridization; RNAse protection assays; and PCR-based methods, such as reverse transcription polymerase chain reaction (RT-PCR) and real time quantitative PCR (also referred to as qRT-PCR). Alternatively, antibodies may be employed that can recognize specific duplexes, including DNA duplexes, RNA duplexes, and DNA-RNA hybrid duplexes, or DNA-protein duplexes. Representative methods for sequencing-based gene expression analysis include Serial Analysis of Gene Expression (SAGE), and gene expression analysis by massively parallel signature sequencing (MPSS).

Some method embodiments involving the determination of mRNA levels utilize RNA (e.g., total RNA) isolated from a target sample, such a prostate cancer tissue sample. General methods for RNA (e.g., total RNA) isolation are well known in the art and are disclosed in standard textbooks of molecular biology.

Differential gene expression also can be determined using microarray techniques. In these methods, specific binding partners, such as probes (including cDNAs or oligonucleotides) specific for RNAs of interest or antibodies specific for proteins of interest are plated, or arrayed, on a microchip substrate. The microarray is contacted with a sample containing one or more targets (e.g., microRNA, mRNA or protein) for one or more of the specific binding partners on the microarray. The arrayed specific binding partners form specific detectable interactions (e.g., hybridized or specifically bind to) their cognate targets in the sample of interest.

In some examples, differential gene expression is determined using in situ hybridization techniques, such as fluorescence in situ hybridization (FISH) or chromogen in situ hybridization (CISH). In these methods, specific binding partners, such as probes labeled with a fluorophore or chromogen specific for a target cDNA, microRNA or mRNA (e.g., a biomarker cDNA or mRNA molecule or microRNA molecule) is contacted with a sample, such as a prostate cancer sample mounted on a substrate (e.g., glass slide). The specific binding partners form specific detectable interactions (e.g., hybridized to) their cognate targets in the sample. For example, hybridization between the probes and the target nucleic acid can be detected, for example by detecting a label associated with the probe. In some examples, microscopy, such as fluorescence microscopy, is used.

A method for detecting biomarkers may include use of biomolecules immobilized on a solid support or substrate in the form of an array. As such, the invention provides an array for detection prostate cancer which includes a plurality of probes for detecting the expression level of a gene selected from those recited in Table 2, Table 3 or Table 6. In various embodiments, the probes are oligonucleotides, polypeptides or antibodies.

As used herein, an array may be any arrangement or disposition of the polypeptides. In one embodiment, the polypeptides are at specific and identifiable locations on the array. Those of skill in the art will recognize that many such permutations of the polypeptides on the array are possible. In another non-limiting embodiment, each distinct location on the array comprises a distinct polypeptide.

Any suitable support or surface may be used. Examples of such supports include, but are not limited to, microarrays, beads, columns, optical fibers, wipes, nitrocellulose, nylon, glass, quartz, diazotized membranes (paper or nylon), silicones, polyformaldehyde, cellulose, cellulose acetate, paper, ceramics, metals, metalloids, semiconductive materials, coated beads, magnetic particles; plastics such as polyethylene, polypropylene, and polystyrene; and gel-forming materials, such as proteins (e.g., gelatins), lipopolysaccharides, silicates, agarose, polyacrylamides, methylmethracrylate polymers; sol gels; porous polymer hydrogels; nanostructured surfaces; nanotubes (such as carbon nanotubes), and nanoparticles (such as gold nanoparticles or quantum dots).

In one embodiment, the support is a solid support. Any suitable “solid support” may be used to which the polypeptides can be attached including but not limited to dextrans, hydrogels, silicon, quartz, other piezoelectric materials such as langasite, nitrocellulose, nylon, glass, diazotized membranes (paper or nylon), polyformaldehyde, cellulose, cellulose acetate, paper, ceramics, metals, metalloids, semiconductive materials, coated beads, magnetic particles; plastics such as polyethylene, polypropylene, and polystyrene; and gel-forming materials, such as proteins (e.g., gelatins), lipopolysaccharides, silicates, agarose and polyacrylamides.

Another aspect of the present invention is that the assay method can be provided in a kit which allows for more convenient laboratory-based biomarker analysis. The kits may include a plurality of components including reagents, supplies, written instructions, and/or software. The kits may have a plurality of embodiments including laboratory kits and mail-in kits. The kits can include secondary reagents. Secondary reagents may be antibodies, enzymes, labels, or chemicals and may enable a complete biomarker panel assay.

Exemplary kits can include at least one means for detection of one or more of the biomarkers (such as, at least two, at least three, at least four, or at least five detection means). In some examples, such kits can further include at least one means for detection of one or more (e.g., one to three) housekeeping genes or proteins. Detection means can include, without limitation, a nucleic acid probe specific for a genomic sequence including a disclosed gene, a nucleic acid probe specific for a transcript (e.g., mRNA) encoded by a disclosed gene, a pair of primers for specific amplification of a disclose gene (e.g., genomic sequence or cDNA sequence of such gene), an antibody or antibody fragment specific for a protein encoded by a disclosed gene.

In some kit embodiments, the primary detection means (e.g., nucleic acid probe, nucleic acid primer, or antibody) can be directly labeled, e.g., with a fluorophore, chromophore, or enzyme capable of producing a detectable product (such as alkaline phosphates, horseradish peroxidase and others commonly known in the art). Other kit embodiments will include secondary detection means; such as secondary antibodies (e.g., goat anti-rabbit antibodies, rabbit anti-mouse antibodies, anti-hapten antibodies) or non-antibody hapten-binding molecules (e.g., avidin or streptavidin). In some such instances, the secondary detection means will be directly labeled with a detectable moiety. In other instances, the secondary (or higher order) antibody will be conjugated to a hapten (such as biotin, DNP, and/or FITC), which is detectable by a detectably labeled cognate hapten binding molecule (e.g., streptavidin (SA) horseradish peroxidase, SA alkaline phosphatase, and/or SA QDot™). Some kit embodiments may include colorimetric reagents (e.g., DAB, and/or AEC) in suitable containers to be used in concert with primary or secondary (or higher order) detection means (e.g., antibodies) that are labeled with enzymes for the development of such colorimetric reagents.

In some embodiments, a kit includes positive or negative control samples, such as a cell line or tissue known to express or not express a particular biomarker.

In some embodiments, a kit includes instructional materials disclosing, for example, means of use of a probe or antibody that specifically binds a disclosed gene or its expression product (e.g., microRNA, mRNA or protein), or means of use for a particular primer or probe. The instructional materials may be written, in an electronic form (e.g., computer diskette or compact disk) or may be visual (e.g., video files). The kits may also include additional components to facilitate the particular application for which the kit is designed. Thus, for example, the kit can include buffers and other reagents routinely used for the practice of a particular disclosed method. Such kits and appropriate contents are well known to those of skill in the art.

Certain kit embodiments can include a carrier means, such as a box, a bag, a satchel, plastic carton (such as molded plastic or other clear packaging), wrapper (such as, a sealed or sealable plastic, paper, or metallic wrapper), or other container. In some examples, kit components will be enclosed in a single packaging unit, such as a box or other container, which packaging unit may have compartments into which one or more components of the kit can be placed. In other examples, a kit includes a one or more containers, for instance vials, tubes, and the like that can retain, for example, one or more biological samples to be tested.

Other kit embodiments include, for instance, syringes, cotton swabs, or latex gloves, which may be useful for handling, collecting and/or processing a biological sample. Kits may also optionally contain implements useful for moving a biological sample from one location to another, including, for example, droppers, syringes, and the like. Still other kit embodiments may include disposal means for discarding used or no longer needed items (such as subject samples). Such disposal means can include, without limitation, containers that are capable of containing leakage from discarded materials, such as plastic, metal or other impermeable bags, boxes or containers.

The kits can further include software. Software may include a training video that may provide additional support including demonstration of biomarker assays, examples of results, or educational materials for performing biomarker assays according to the invention.

The following examples are provided to further illustrate the embodiments of the present invention, but are not intended to limit the scope of the invention. While they are typical of those that might be used, other procedures, methodologies, or techniques known to those skilled in the art may alternatively be used.

### Example 1

### Integrated RNA and Metabolite Profiling of Urine Liquid Biopsies for Prostate Cancer Biomarker and Target Discovery

Sensitive and specific diagnostic and prognostic biomarkers for prostate cancer (PCa) are urgently needed. Urine samples are a non-invasive means to obtain abundant and readily accessible “liquid biopsies”. Herein, urine liquid biopsies were used to identify and characterize a novel group of urine-enriched RNAs and metabolites in PCa patients and normal individuals with or without benign prostatic disease. Differentially expressed RNAs were identified in urine samples by deep sequencing. Amino acids, acylcarnitines, and organic acids in urine were measured by mass spectrometry. The mRNA and metabolite profiles were distinct in patients with benign and malignant disease. Integrated analysis of urinary gene expression and metabolite signatures unveiled a glutamate (Glu) metabolism and tricarboxylic acid (TCA) cycle node that was aberrant in prostate-derived cancer cells. Functional validation supports a role for Glu metabolism and glutamate oxaloacetate transaminase 1 (GOT1)-dependent redox balance in prostate cancer which can be exploited for novel biomarkers and therapies.

The following methods were utilized in this study.

Sample Collection and Preparation

Urine samples were collected from 20 benign prostatic hyperplasia (BPH), 11 prostatitis, 20 prostate cancer (PCa) patients and 20 normal healthy individuals at the Global Robotics Institute (Celebration, Fla., USA) and Florida Urology Associates (Orlando, Fla., USA) of Florida Hospital between 2008-2014. The institutional review board of the Florida Hospital approved the use of samples. All participants were required to sign and provide written consent forms. Urines were collected using urine preservation tubes (Norgen Bioteck, Thorold, ON, Canada) and kept at room temperature. The exfoliated cells in urine samples were removed by centrifugation and cell-free urine was stored at −80° C. until use for metabolite analysis. The exfoliated cells from normal and PCa urine samples were used for total RNA purification using the Urine (exfoliated cell) RNA purification kit (Norgen Bioteck).

Global Metabolomics

Global metabolomics was performed by liquid chromatography high resolution mass spectrometry (LC-HRMS) on a Thermo-Q-Exactive™ with Dionex™ UHPLC (Thermo Fisher Scientific, San Jose, Calif.). To 50 μL of urine, 20 μL of internal standard was added (tryptophan D3, leucine-D10, creatine-D3, and caffeine-D3) followed by 400 μL of 98:2 acetonitrile:water with 0.1% sodium azide. The solution was vortex mixed and then spun down at 20,000×g (8C) for 10 min. The supernatant was transferred to a new microcentrifuge tube and dried under a gentle stream of nitrogen. The dried sample was reconstituted in 50 μL of 0.1% formic acid in water and transferred to a LC vial with fused glass insert for analysis. LC-HRMS analysis was performed in positive and negative ion modes as separate injections, injecting 2 μL for positive and 4 μL for negative ions. Separation was achieved on a C18-pfp column (ACE Excel 100×2.1 mm, 2 μm, Advanced Chromatography Technologies, Aberdeen, Scotland) with 0.1% formic acid in water as A and acetonitrile at B. Metabolites were identified by matching to an in house retention time library of 600 metabolites.

Targeted Metabolomics Analysis

Targeted metabolomics of urine was performed at the Proteomics Core Facility at Sanford Burnham Prebys Medical Research Institute (SBPMRI) (Orlando, Fla., USA). Frozen cell-free urine samples were thawed and prepared for analysis according to the core's proprietary solvent extraction methods. The prepared samples were subjected to analysis on LC/MS/MS platforms. Authentic heavy isotope-labeled internal standards were used for all amino acids (excluding 1- and 3-methylhistidine, which utilize histidine's internal standard), all organic acids, and C2, C3, C4 butyryl, C5 isovaleryl, C6, C8, C10, C12, C14, C16, and C18 acylcarnitines.

Cell Culture

Prostate cancer cell lines LNCaP (ATCC® CRL-1740™) and PC3 (ATCC® CRL-7934™) were cultured in RPMI1640 medium and Dulbecco's Modified Eagle Medium (Thermo Fisher Scientific Inc., Waltham, Mass.), respectively, supplemented with 10% FBS and penicillin/streptomycin.

RNA Isolation, cDNA Synthesis, and Quantitative Real-Time PCR (qPCR)

Total RNAs from cell lines were purified using the Direct-zol RNA Miniprep™ kit (Zymo Research, Irvine, Calif.). Normal prostate epithelial cell RNA was purchased from BioChain Institute Inc. (Catalog # R1234201-50, Newark, Calif.). RNA (0.5 ug) was then used for cDNA synthesis using a high capacity cDNA reverse transcription kit (Applied Biosystems, Foster City, Calif.). qPCR was performed using a Power SYBR™ Green PCR master mix (Applied Biosystems, Warrington, UK) in the 7500 Real-Time™ PCR system (Applied Biosystems). A final reaction volume of 10 ul was used containing 1 ul (corresponding to 10 ng) of cDNA template, 5 ul of 2× Power SYBR™ Green PCR master mix (Applied Biosystems), and 0.2 uM of each primer. The reaction was subjected to denaturation at 95° C. for 10 min followed by 40 cycles of denaturation at 95° C. for 15 sec and annealing at 58° C. for 1 min. SDS1.2.3 software (Applied Biosystems) was used for comparative Ct analysis with TATA-box binding protein (TBP) serving as the endogenous control. The primer sequences for the genes were listed in Table 1.

TABLE 1
Primer Sequences.

| | | Ampli- | |
|---|---|---|---|
| | | con | SEQ |
| | | size | ID |
| Oligos | Sequence | (bp) | NO |
| BRD3 qPCR F | CTGAAACCCACCACTTTGCG | 84 | 1 |
| BRD3 qPCR R | GCTTGCTGAGAACGGTTTCC | | 2 |
| ELK4 qPCR F | GCCCCTTGCTCTCCAGTATC | 147 | 3 |
| ELK4 qPCR R | CATCCAGCCCAGACAGAGTG | | 4 |
| EPCAM qPCR F | GTTCGGGCTTCTGCTTGC | 89 | 5 |
| EPCAM qPCR R | CAGTTTACGGCCAGCTTGTA | | 6 |
| FH qPCR F | CCGAGCACTTCGGCTCCT | 150 | 7 |
| FH qPCR R | ATCCGGAAGGAATTTTGGCTTG | | 8 |
| GRHPR qPCR F2 | GACCACGTGGACAAGAGGAT | 146 | 9 |
| GRHPR qPCR R2 | TCTGTCAGGACATCTGGGGT | | 10 |
| HDAC6 qPCR F | GCTGACTACCTAGCTGCCTG | 89 | 11 |
| HDAC6 qPCR R | TCAAAGCCAGCTGAGACCAG | | 12 |
| NACA qPCR F | CACGCTCTCGCTCGGTCTTT | 148 | 13 |
| NACA qPCR R | GGCTGAAGACATAGGTAGCACA | | 14 |
| NPM1 qPCR F | ACTCCAGCCAAAAATGCACA | 208 | 15 |
| NPM1 qPCR R | TACATGTAGTGCCCAGGACTGTT | | 16 |
| PHB qPCR F | ATCACCCAGAGAGAGCTGGT | 132 | 17 |
| PHB qPCR R | CACCGCTTCTGTGAACTCCT | | 18 |
| RPL22 qPCR F | TGACATCCGAGGTGCCTTTC | 101 | 19 |
| RPL22 qPCR R | GTTAGCAACTACGCGCAACC | | 20 |
| RPS11 qPCR F | GTACACCTGTCCCCCTGCTT | 91 | 21 |
| RPS11 qPCR R | TGAAGCGCACTGTCTTGCT | | 22 |
| SMARCB1 qPCR F2 | GCGAGTTCTACATGATCGGCT | 145 | 23 |
| SMARCB1 qPCR R2 | CCGTGATCATGTGACGATGC | | 24 |
| TFG qPCR F | ATCGTTCAGGAACACCCGAC | 147 | 25 |
| TFG qPCR R | CCTGTTGCTGGTACTGTTGG | | 26 |
| GOT1 qPCR F | AGAAGCCCTCAAAACCCCTG | 143 | 27 |
| GOT1 qPCR R | CGTTGATTCGACCACTTGGC | | 28 |
| HPRT1 E2 qPCR F | TGCTGAGGATTTGGAAAGGGT | 99 | 29 |
| HPRT1 E3 qPCR R | TGATGGCCTCCCATCTCCTT | | 30 |
| TBP E2 qPCR F | ACAACAGCCTGCCACCTTAC | 96 | 31 |
| TBP E3 qPCR R | TGCCATAAGGCATCATTGGACT | | 32 |
| ACTB qPCR F | CCTGGCATTGCCGACAGGATG | 107 | 33 |
| ACTB qPCR R | CCGATCCACACGGAGTACTTGCG | | 34 |

RNA Access

The quantity and integrity of the RNA was measured using both the Qubit RNA HS™ Assay Kit (Thermo Fisher Scientific Inc.) and the Agilent 2100 Bioanalyzer™ RNA Pico kit (Agilent Technologies, Santa Clara, Calif.). Following the Illumina DV₂₀₀ metric (percentage of RNA fragments greater than 200 nucleotides), 100 ng of RNA with DV₂₀₀>30% was used to prepare sequencing libraries in accordance with the TruSeq RNA Access protocol (Illumina, Inc., San Diego, Calif.). First strand cDNA was synthesized using random primers followed by second strand synthesis. The cDNA then underwent 3′ adenylation followed by adapter ligation and PCR amplification (15 cycles). The quality of the libraries was measured using both the Qubit dsDNA HS' Assay Kit and Agilent Bioanalyzer™ DNA kit. A 4-plex pool of libraries were then made (200 ng each sample) followed by two rounds of hybridization/capture and a final amplification (10 cycles). The quality and quantity of the final libraries were determined by the Agilent 2100 Bioanalyzer DNA HS™ kit and Kapa Biosystems™ qPCR (Kapa Biosystems, Inc., Wilmington, Mass.). Multiplexed libraries were pooled and normalized to 17.5 pM. The libraries were sequenced using a 75 bp paired-end run on the Illumina MiSeg™ instrument. Paired-end reads were mapped to the human genome (hg19) using tophat2.0.1™; mapped reads were filtered based on the mapping quality. The overall mapping rates were about 93%. mRNA quantification was done using Partek Genomics™ Suite 6.6. R package edgeR was used to analyze the differential expression of mRNAs.

Clustering and Principal Component Analysis (PCA)

The resulting mRNA expression profile included 46,459 transcripts, and the non-parametric Mann-Whitney U-test was used to identify significantly regulated transcripts. 5510 transcripts were identified (p<0.05) as significantly differentially expressed between normal and PCa groups. Within those 5,510 transcripts, 1,118 transcripts had RPKM values greater than 1.0 for all samples. A gene panel was pre-compiled that lists all prostate cancer-related genes (with the help of Illumina). Comparing with this panel, 542 transcripts were obtained with RPKMs greater than 1.0 for all samples. Within these 542, 116 transcripts were significantly regulated (Tables 2 (Upregulated) and 3 (Downregulated)).

TABLE 2
Upregulated Genes.

| | Wilcoxon p-value | |
|---|---|---|
| Gene | Upregulated | log2 fold-change |
| ACE | 0.04 | 1.0 |
| ATF1 | 0.01 | 0.6 |
| BRD3 | 0.03 | 0.2 |
| CCNB1IP1 | 0.01 | 0.4 |
| CDC14A | 0.03 | 0.3 |
| CDK8 | 0.01 | 0.2 |
| ELK4 | 0.02 | 0.9 |
| EPCAM | 0.04 | 0.4 |
| FH | 0.04 | 0.0 |
| GMPS | 0.01 | 0.2 |
| GNAS | 0.02 | 0.8 |
| GOT1 | 0.02 | 0.5 |
| GRHPR | 0.00 | 1.3 |
| HDAC6 | 0.04 | 0.6 |
| HSP90AB1 | 0.04 | 0.6 |
| LRPPRC | 0.02 | 0.7 |
| MSH3 | 0.00 | 0.1 |
| NACA | 0.03 | 1.1 |
| NPM1 | 0.01 | 2.8 |
| PFDN5 | 0.02 | 0.7 |
| PHB | 0.01 | 0.3 |
| PHF6 | 0.04 | 0.2 |
| PIK3R1 | 0.00 | 1.4 |
| PTK2 | 0.03 | 1.1 |
| PTPN2 | 0.02 | 2.8 |
| RPL22 | 0.01 | 0.9 |
| RPS11 | 0.01 | 0.9 |
| SDHC | 0.02 | 2.9 |
| SDHD | 0.03 | 1.0 |
| SMAD4 | 0.02 | 0.0 |
| SMARCB1 | 0.00 | 0.3 |
| TCEA1 | 0.03 | 0.1 |
| TERF2 | 0.02 | 0.3 |
| TFG | 0.03 | 0.1 |
| TMEM230 | 0.02 | 0.2 |
| ZMYND11 | 0.00 | 3.1 |
| ZNF585B | 0.02 | 0.4 |

TABLE 3
Downregulated Genes.

| | Wilcoxon p-value | |
|---|---|---|
| Gene | Downregulated | log2 fold-change |
| ACSL3 | 0.02 | −1.3 |
| ADM | 0.04 | −2.5 |
| BAZ2A | 0.01 | −0.8 |
| BCL10 | 0.00 | −2.3 |
| BIRC3 | 0.04 | −4.1 |
| BRWD3 | 0.00 | −1.5 |
| CDKL5 | 0.01 | −1.1 |
| CDKN1B | 0.02 | −1.4 |
| CHIC2 | 0.01 | −1.8 |
| CIC | 0.02 | −3.9 |
| CREBBP | 0.03 | −1.2 |
| DDB2 | 0.03 | −1.3 |
| DOT1L | 0.01 | −1.2 |
| ELF4 | 0.02 | −1.8 |
| EP300 | 0.01 | −0.8 |
| EPHA2 | 0.00 | −3.2 |
| ERBB3 | 0.04 | −0.6 |
| ERCC5 | 0.03 | −0.9 |
| ETV6 | 0.04 | −1.2 |
| FLCN | 0.00 | −1.0 |
| FOSB | 0.03 | −2.1 |
| GIT2 | 0.04 | −5.1 |
| GNAQ | 0.02 | −1.0 |
| HDAC4 | 0.02 | −1.3 |
| HIPK1 | 0.03 | −1.6 |
| HIST1H2BO | 0.04 | −0.9 |
| ICAM1 | 0.03 | −3.1 |
| IGF1R | 0.04 | −1.1 |
| IRF1 | 0.03 | −2.1 |
| JAK2 | 0.04 | −0.8 |
| KDM5A | 0.04 | −0.1 |
| KDM5C | 0.01 | −1.3 |
| KIAA0232 | 0.01 | −1.0 |
| KMT2B | 0.03 | −0.7 |
| KMT2C | 0.03 | −0.9 |
| KMT2D | 0.02 | −0.9 |
| LAMP2 | 0.02 | −2.9 |
| LMO7 | 0.00 | −3.0 |
| LRMP | 0.02 | −3.2 |
| MALAT1 | 0.00 | −3.2 |
| MCL1 | 0.03 | −4.0 |
| MDM2 | 0.03 | −1.2 |
| MYD88 | 0.02 | −4.2 |
| NCSTN | 0.02 | −1.7 |
| NDE1 | 0.02 | −2.0 |
| NIPBL | 0.02 | −1.3 |
| NOTCH1 | 0.01 | −2.9 |
| NT5C2 | 0.02 | −2.0 |
| NUP98 | 0.01 | −2.3 |
| PER1 | 0.00 | −1.9 |
| PICALM | 0.04 | −2.4 |
| PICALM | 0.01 | −3.0 |
| PLCG2 | 0.03 | −1.6 |
| PRDM1 | 0.00 | −2.6 |
| PRSS8 | 0.00 | −3.3 |
| PTK2B | 0.00 | −2.1 |
| RAF1 | 0.04 | −1.6 |
| RCOR1 | 0.00 | −1.3 |
| RNF213 | 0.03 | −1.5 |
| SH3BP1 | 0.01 | −1.6 |
| SMAD3 | 0.02 | −1.4 |
| STRN | 0.02 | −0.3 |
| TMEM127 | 0.02 | −1.9 |
| TMPRSS2 | 0.02 | −2.0 |
| TNFAIP3 | 0.03 | −2.2 |
| TOP1 | 0.02 | −1.3 |
| TPM4 | 0.02 | −2.2 |
| TYK2 | 0.02 | −1.9 |
| VHL | 0.04 | −1.8 |
| WDFY3 | 0.04 | −1.0 |
| WDR1 | 0.04 | −1.7 |
| WSB1 | 0.03 | −1.7 |
| XIAP | 0.00 | −1.4 |
| ZNF217 | 0.00 | −1.1 |

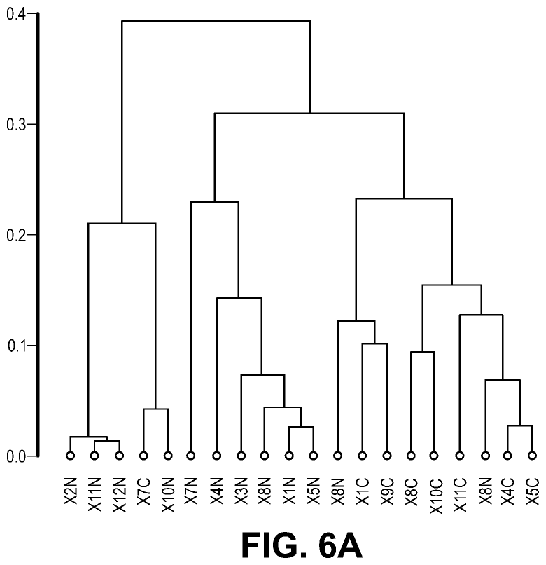

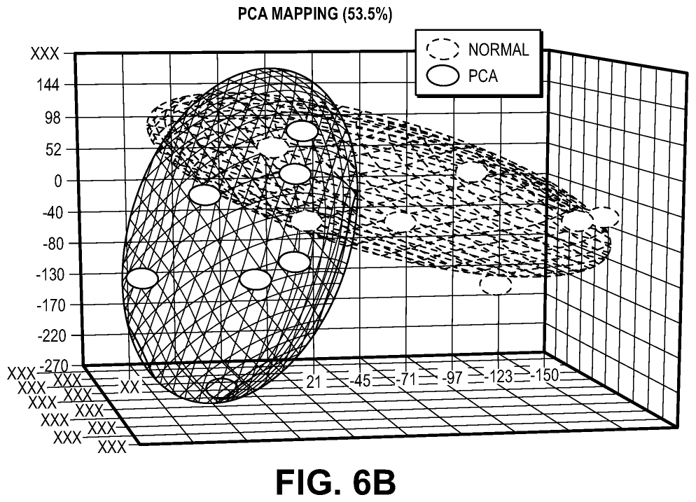

All overlapping transcripts between the expression profile and prostate cancer panel, a total of 3825 transcripts, were used to run the unsurprised clustering analysis and PCA. Hierarchical cluster analysis was performed in R using the correlation between samples to characterize similarity. Initially, each sample was assigned to its own cluster and then the algorithm proceeded iteratively, at each stage joining the two most similar clusters, continuing until there was just a single cluster. Correlation between samples was calculated using the expression values of 3825 transcripts. PCA was also used to visualize sample to sample distance. The transformation was defined that the first principal component accounted for the largest variance (as much of the variability in the dataset as possible). In the results, each sample was projected onto the 3D space in which the three axes were the first three highest principle components (see FIG. 6B).

Transient Transfection and Cell Proliferation Assay

Cells (0.3×10⁶ cells) were mixed with siRNA (Thermo Fisher Scientific, final concentration 20 nM) and lipofectamine RNAiMAX™ (Thermo Fisher Scientific) mixture in 2 ml media containing 10% FBS. Cells were plated in duplicate at 7500 cells per well into 96-well plates and, after 24 h, medium was replaced. Cell proliferation was assessed using the CellTiter96™ Aqueous One Solution Cell Proliferation Assay (MTS) kit (Promega, Madison, Wis.).

NAD and NADH quantification

NAD and NADH levels were measured using NAD/NADH-Glo™ Assay kit (Promega). Cells were plated in duplicate (7500 cells per well) into 96-well plates. Cells were lysed with base solution (100 mM sodium carbonate, 20 mM sodium bicarbonate, 10 mM nicotinamide, and 0.05% Triton X-100) with 1% (DTAB, Sigma, D8638). Lysates were heat-treated at 60° C. for 20 min in the presence/absence of acid. Heat-treated samples were then subjected to the luciferase assay according to the manufacturer's protocol and the NAD/NADH ratio was calculated.

Measurement of Reactive Oxygen Species (ROS)

Cells (1×10⁶ cells) were resuspended in 1 ml media containing 20 uM 2′7′-dichlorofluoresicin diacetate (DCFH-DA) (Sigma, D6883) and incubated for 30 min at 37° C. in 5% CO₂. Fluorescent cells were detected using a FACS Calibur™ flow cytometer (Becton Dickinson, Anaheim, Calif.) and data were analyzed with the Software 2.5.1™ from Flowing Software Corporation.

Invasion Assay

The cell invasion assay was performed using Corning BioCoat™ Matrigel Invasion Chambers (Discovery Labware, Bedford, Mass.) according to the manufacturer's protocol. Briefly, cells were starved in serum-free medium for 24 h and plated into the upper chambers in serum-free medium (0.2×10⁶ cells per chamber). Medium containing 10% FBS was added to the lower chambers. Cells were incubated for 48 h at 37° C. Invaded cells were stained with 0.5% crystal violet dye. After washing the excess dye, cells were air dried. Methanol was used to extract dye from cells and optical density was measured at 570 nm.

Soft Agar Colony Formation Assay

To access anchorage-independent growth, a CytoSelect™ 96-well cell transformation assay kit (Cell Biolabs Inc., San Diego, Calif.) was used according to the manufacturer's protocol. Briefly, cells were seeded in soft agar at a density of 10,000 cells per well and incubated at 37° C. in 5% CO₂ for 7 days. Colony formation was quantified by the MTT assay provided with the kit according to the assay protocol.

### FIGURE LEGENDS

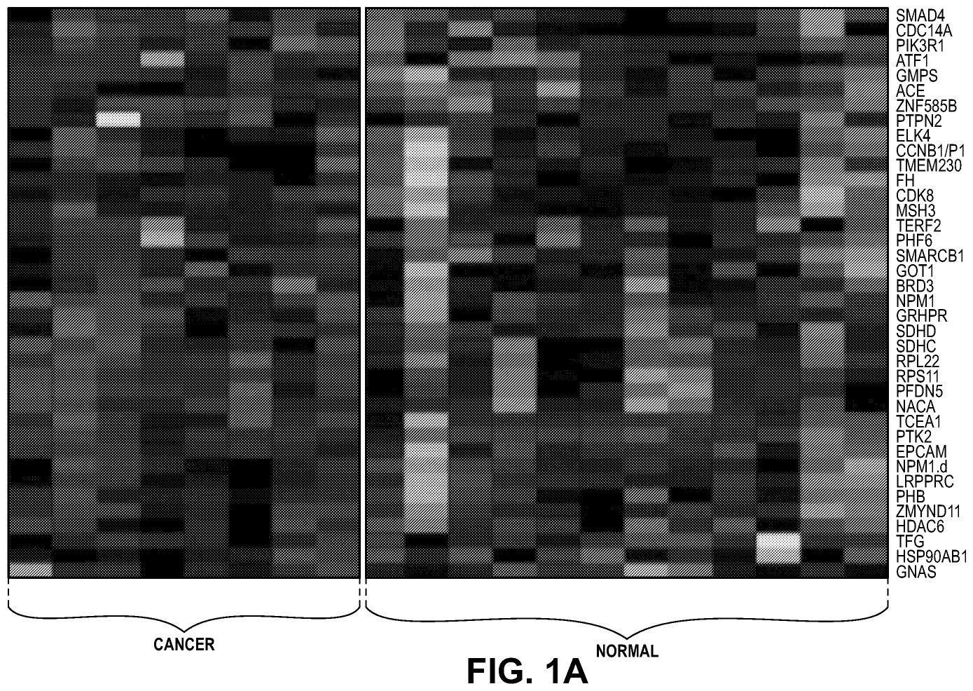

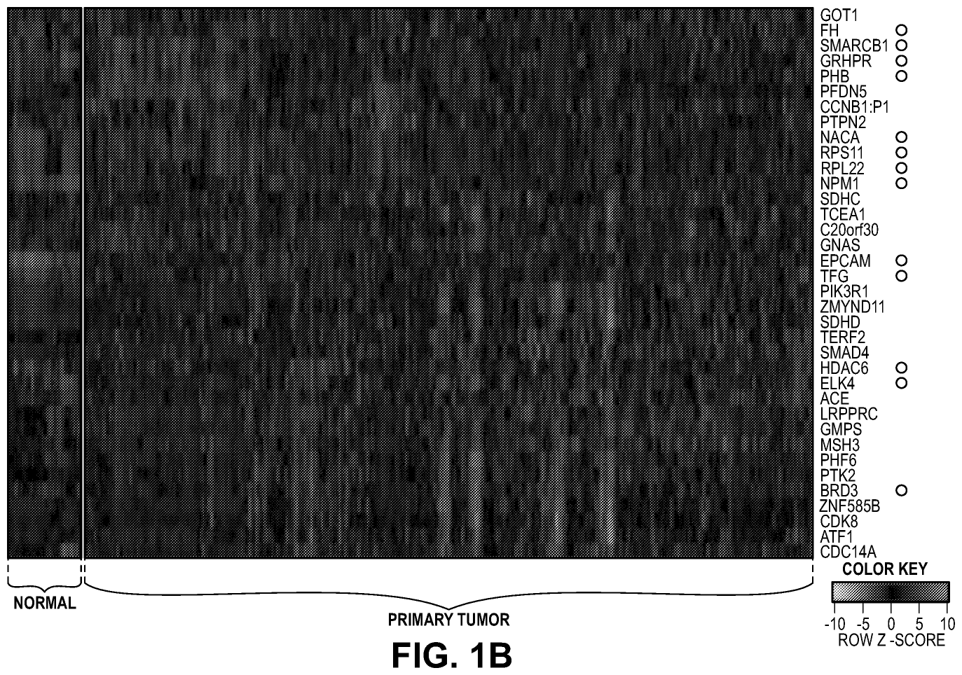

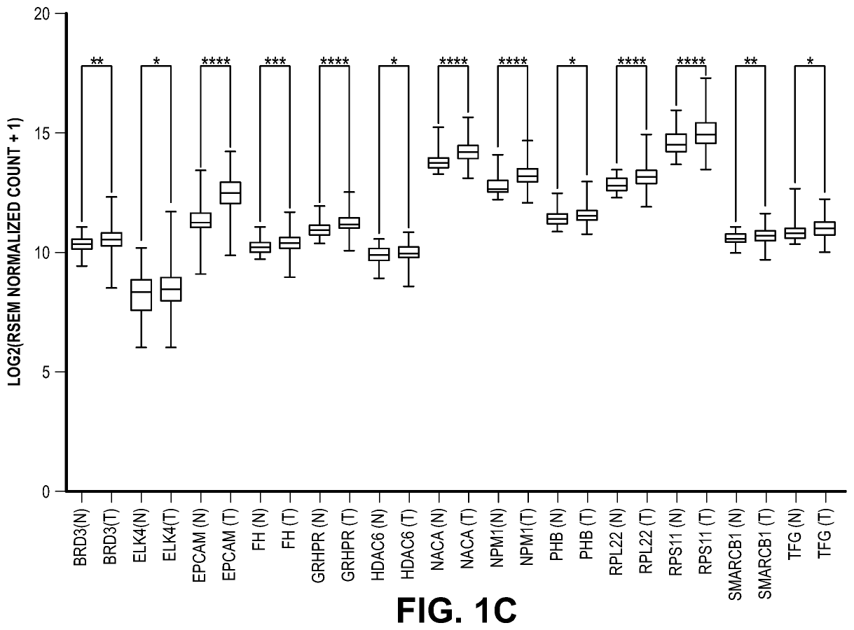

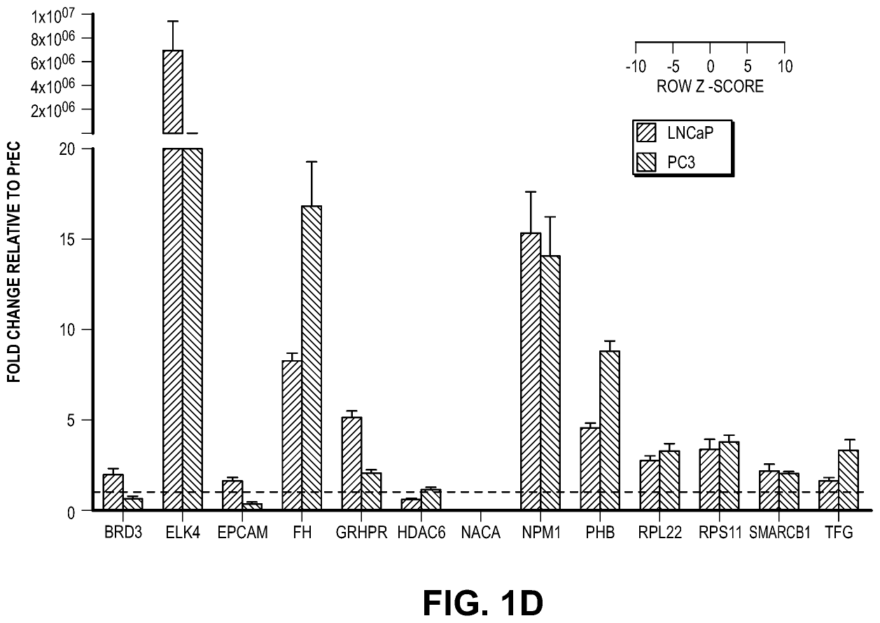

FIG. 1. RNA-seq of cells extracted from the urine of patients with (n=8) and without (n=12) prostate cancer (A) and validation of their differential expression in The Cancer Genome Atlas (TCGA) data (B, C). Among 37 upregulated genes, 13 genes (FH, SMARCB1, GRHPR, PHB, NACA, RPS11, RPL22, NPM1, EPCAM, TFG, HDAC6, ELK4, BRD3) were significantly upregulated in primary tumors (n=497) compared to normal (n=52) in TCGA data. In the heat map, black dots next to the gene name marks the genes upregulated in primary tumor compare to normal in TCGA data. The TCGA project for PCa data is publicly available for download on the World Wide Web at portal.gdc.cancer.gov/projects/TCGA-PRAD. These 13 genes were also tested in two prostate cancer cell lines (LNCaP and PC3, (D). Most were overexpressed apart from NACA, which was down-regulated in both cell lines, BRD3 and EPCAM, which were decreased in PC3 cells, and HDAC6, which was downregulated in LNCaP cells.

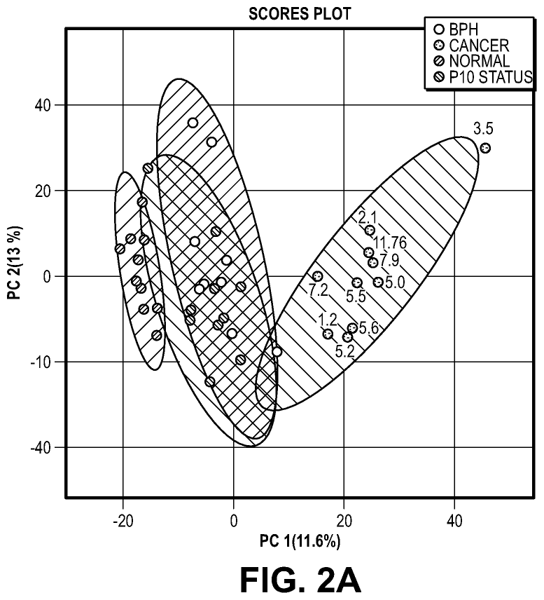

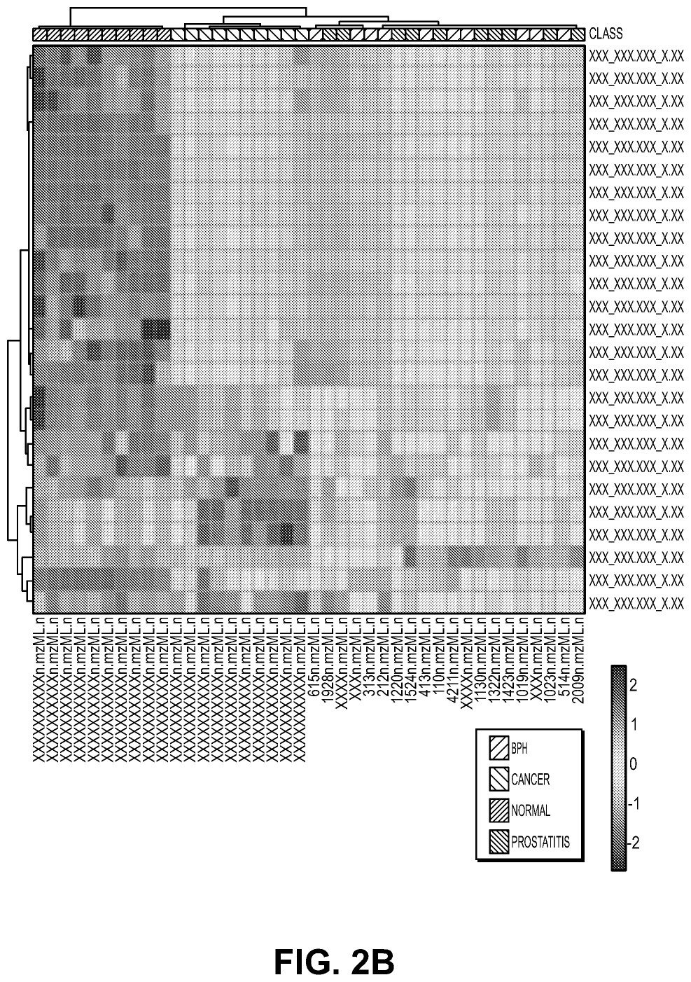

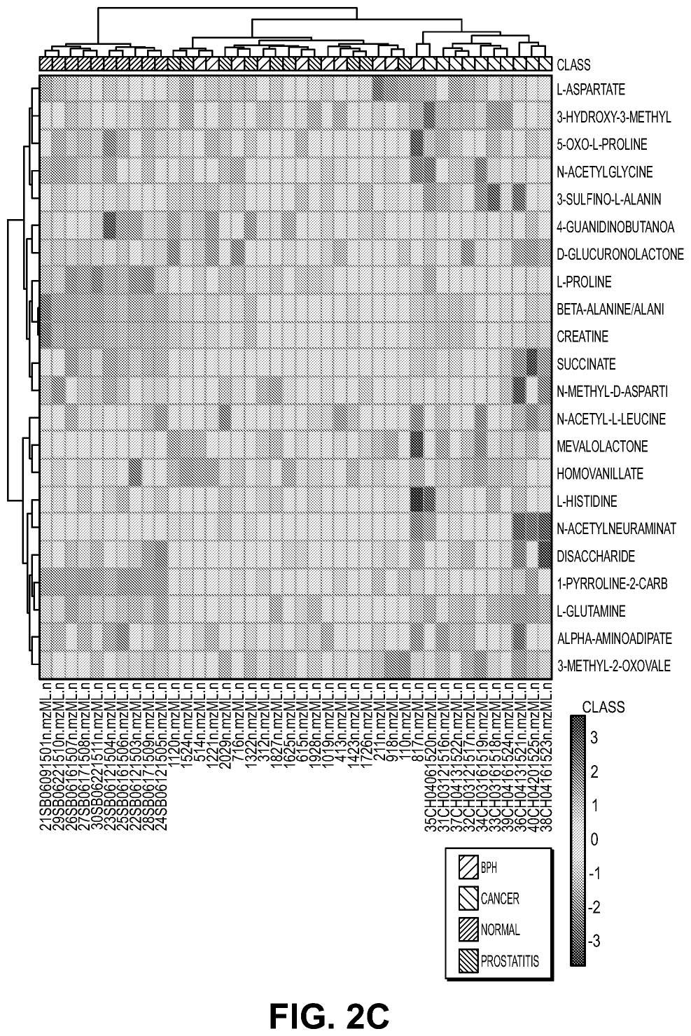

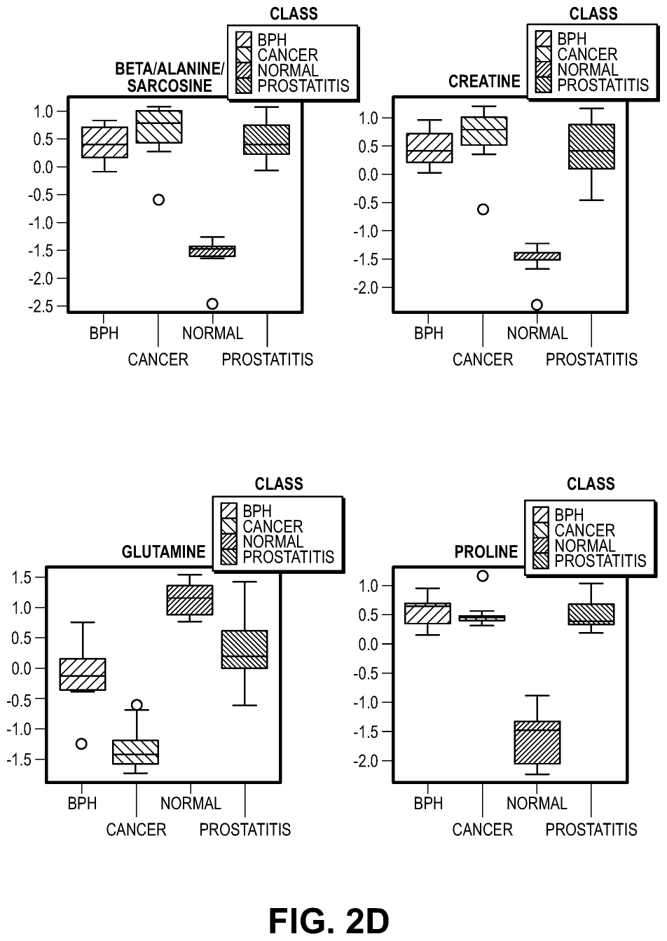

FIG. 2. Metabolic signatures in the urine of normal individuals and patients with BPH, prostatitis, and prostate cancer. (A) PCA plots showing the distinct and separate metabolic profiles of normal urine and urine from patients with cancer but significant overlap between the profiles obtained from patients with BPH and prostatitis. (B) A heat map using only the top 25 metabolites from ANOVA. A heat map (C) using only top 22 known metabolites from ANOVA and 4 representative box plots (D) showing the individual expression in each group.

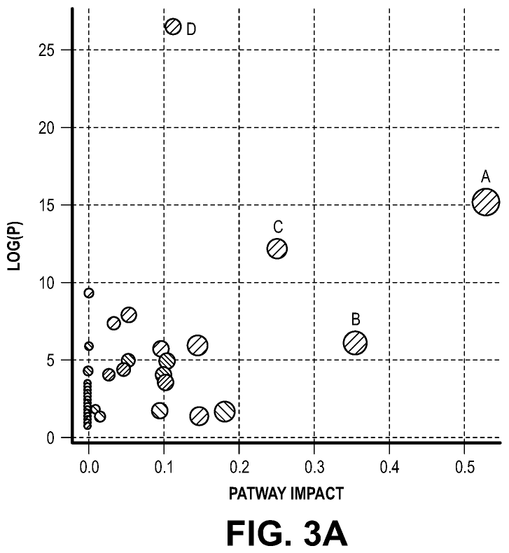

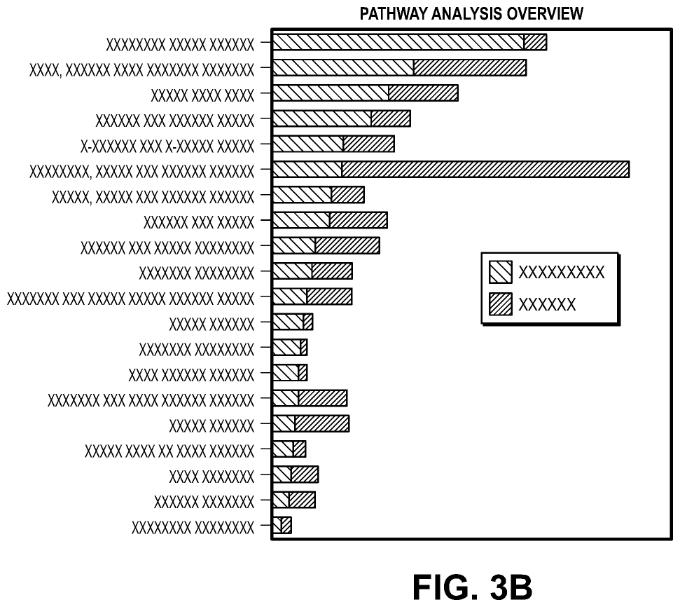

FIG. 3. Metabolic pathways represented by altered metabolites in the urine of normal and PCa patients. (A) Pathway enrichment and predicted impact of up- or downregulated metabolites in the urine of patients with prostate cancer compared to normal: (a) alanine, aspartate and glutamate metabolism; (b) D-glutamine and D-glutamate metabolism; (c) arginine and proline metabolism; (d) aminoacyl-tRNA biosynthesis. (B) Integrative pathway analysis of gene expression and metabolic changes in the urine of normal vs. cancer patients.

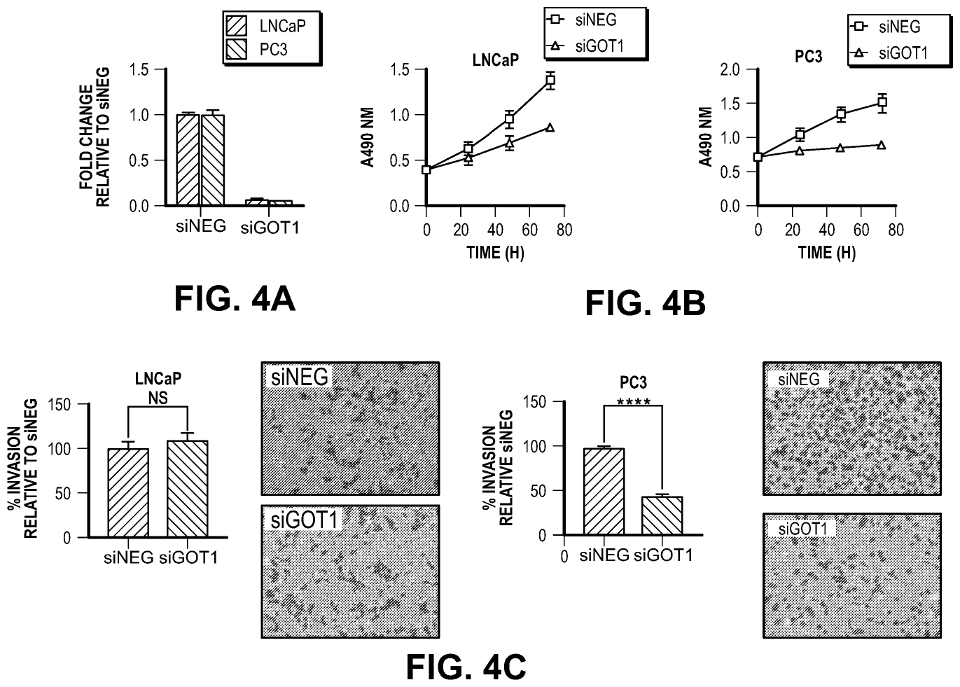

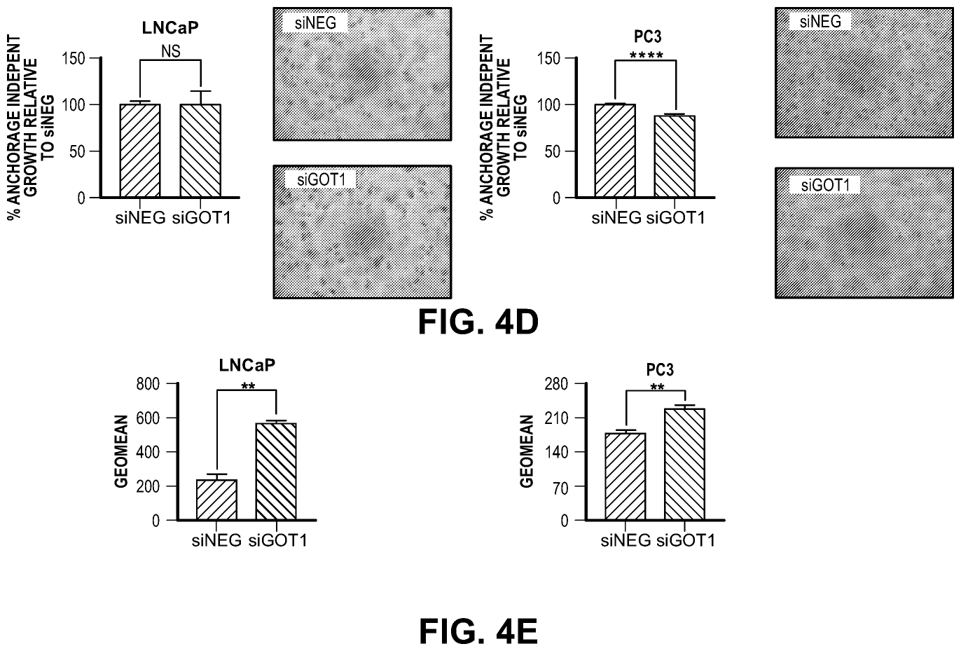

FIG. 4. GOT1 supports proliferation, invasion, and colony formation in prostate cancer cell lines. (A) GOT1 knockdown in LNCaP and PC3 prostate cancer cell lines. (B) GOT1 knockdown significantly inhibits cell viability in PC3 and LNCaP cells. (C) Invasion and (D) anchorage-independent growth in prostate cancer cell lines upon GOT1 knockdown. (E) GOT1 knockdown significantly increases ROS production in PC3 and LNCaP cells. The data from three independent experiments were expressed as mean±SD.

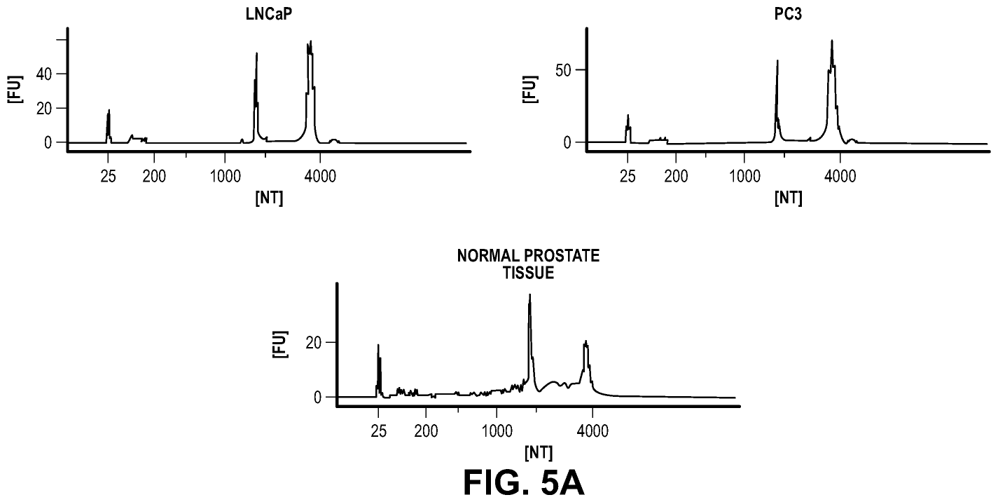

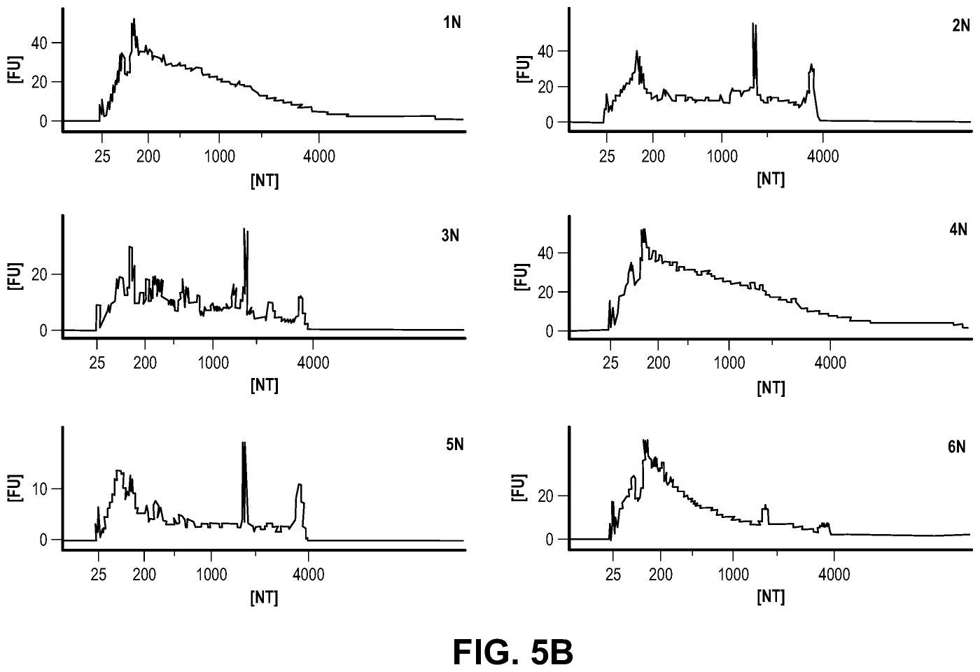

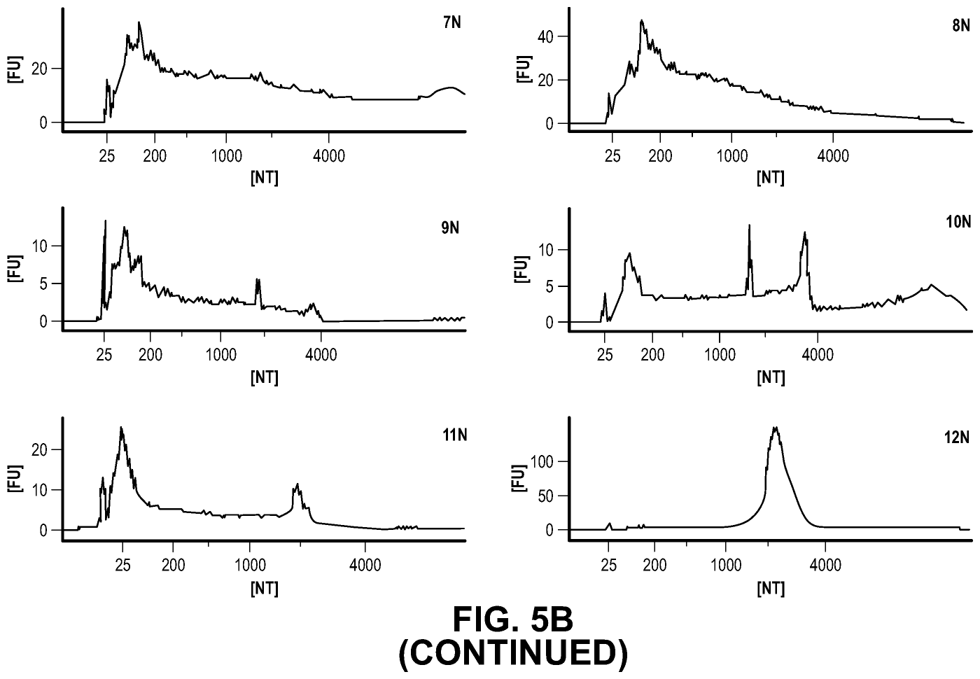

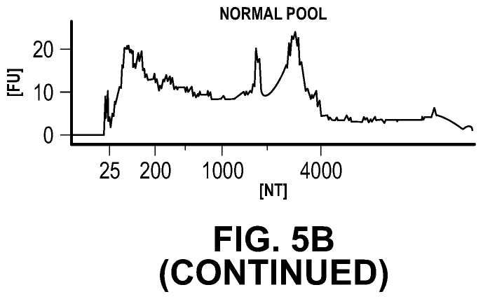

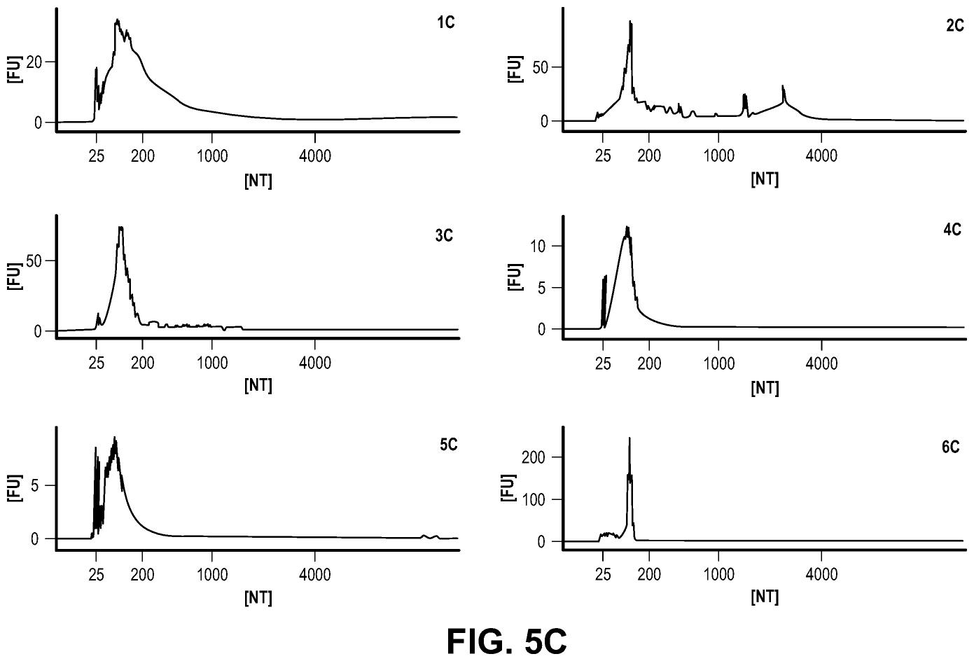

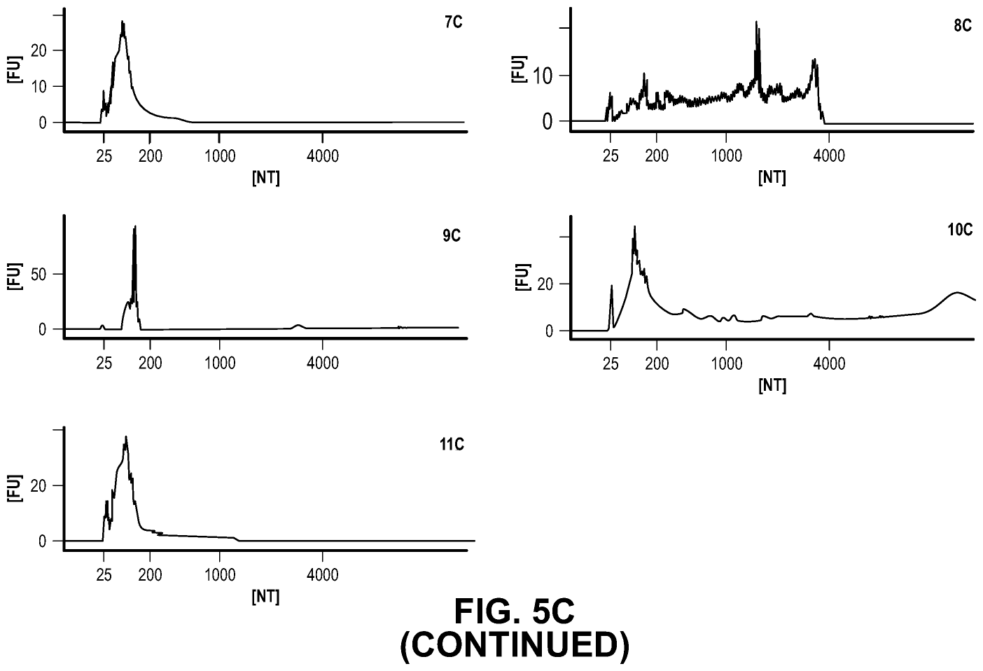

FIG. 5. Electropherograms of RNA extracted from (A) LNCaP and PC3 prostate cancer cell lines and normal prostate tissue (control); (B) RNA from the urine of “normal” patients; and (C) RNA from the urine of patients with cancer. RNA extracted from urine is generally severely degraded.

FIG. 6. Unsupervised clustering (using Treeview; A) and principal component analysis (PCA; B) of significantly differentially expressed genes detected in the cells extracted from urine of normal (green) and cancer (red) patients. Normal and cancer specimens are readily but not perfectly separated.

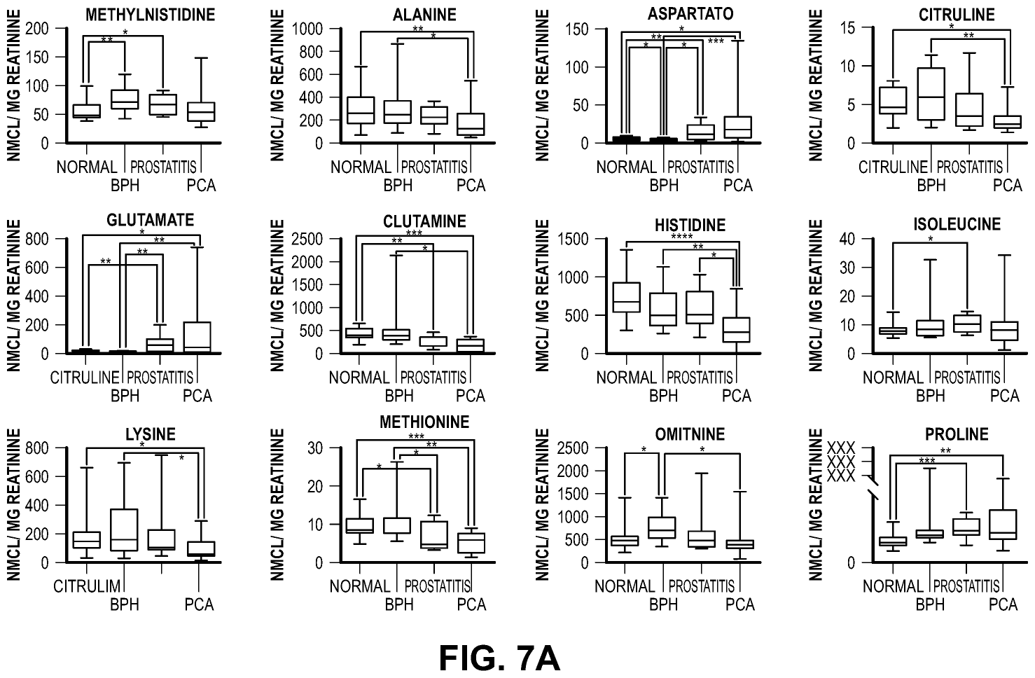

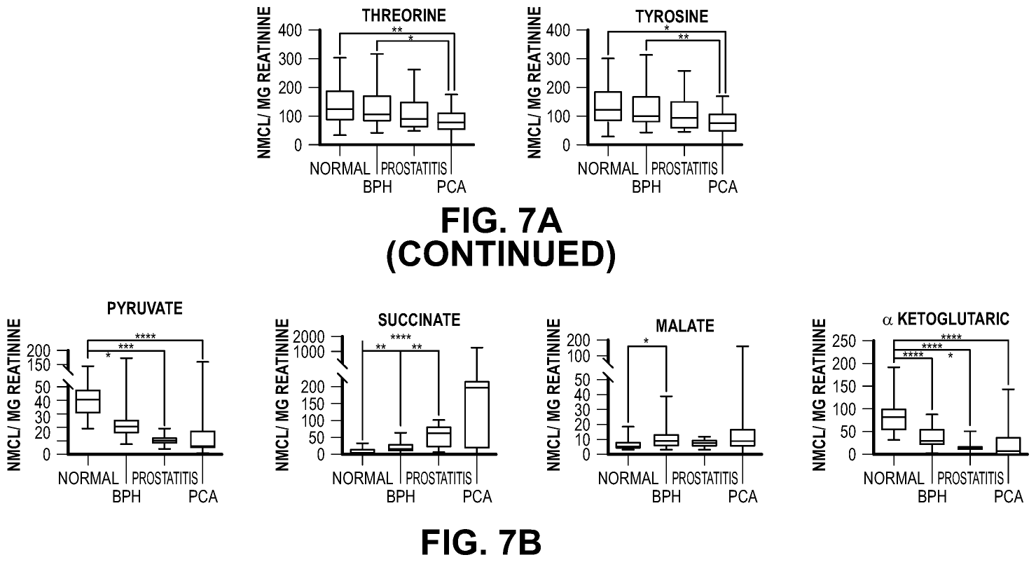

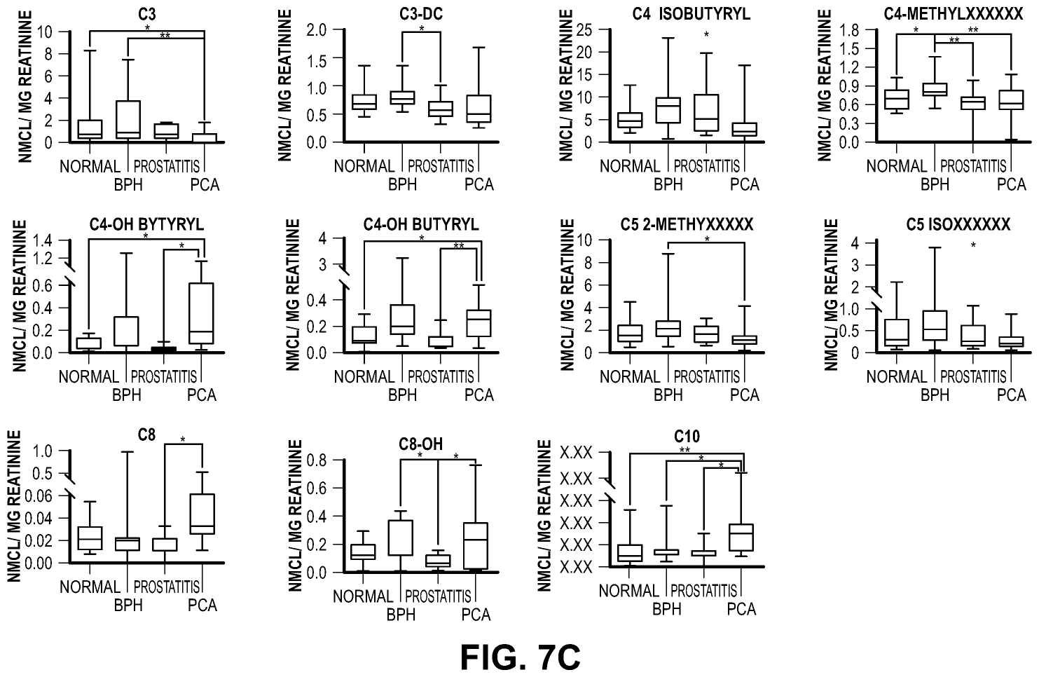

FIG. 7. Metabolites showing significantly different levels between clinical groups.

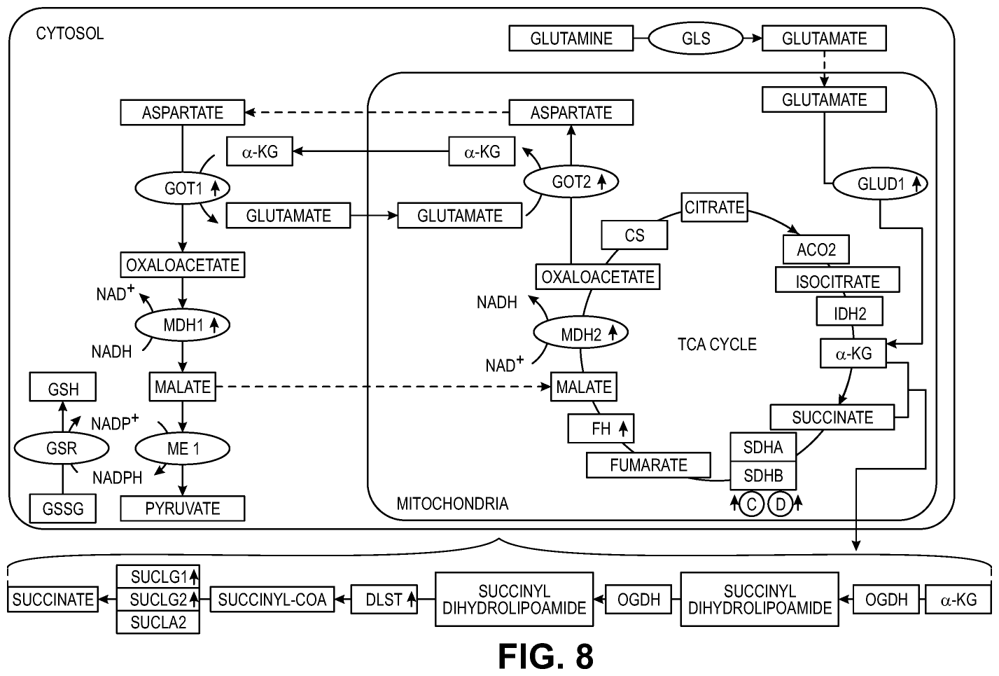

FIG. 8. Schematic view of TCA cycle and glutamine metabolism. Red arrows indicate up-regulated genes in PCa urine compared to normal urine samples.

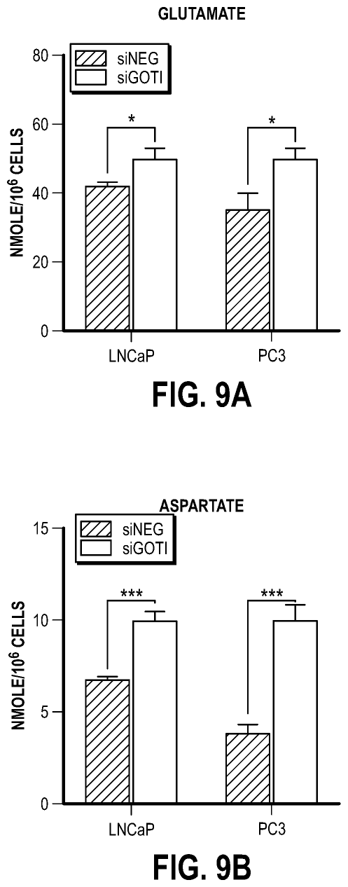

FIG. 9. Glutamate and Aspartate level changes in GOT1 knock down LNCaP and PC3 prostate cancer cell lines. The data from three independent experiments were expressed as mean±SD.

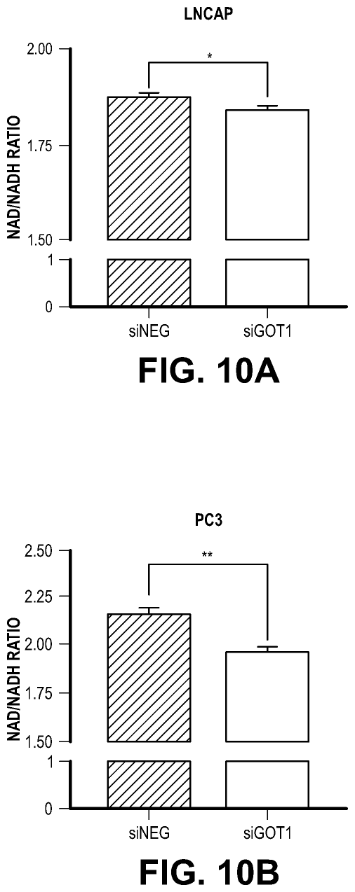

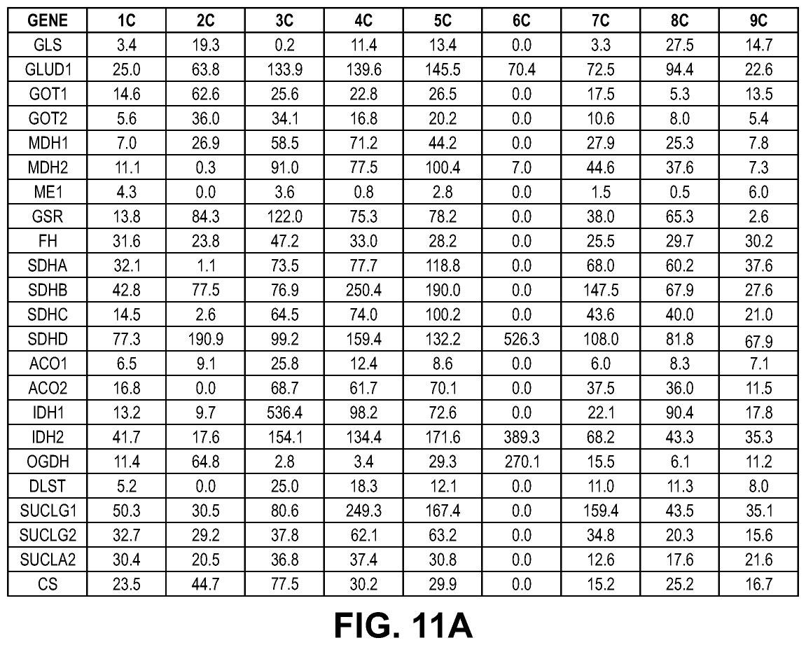

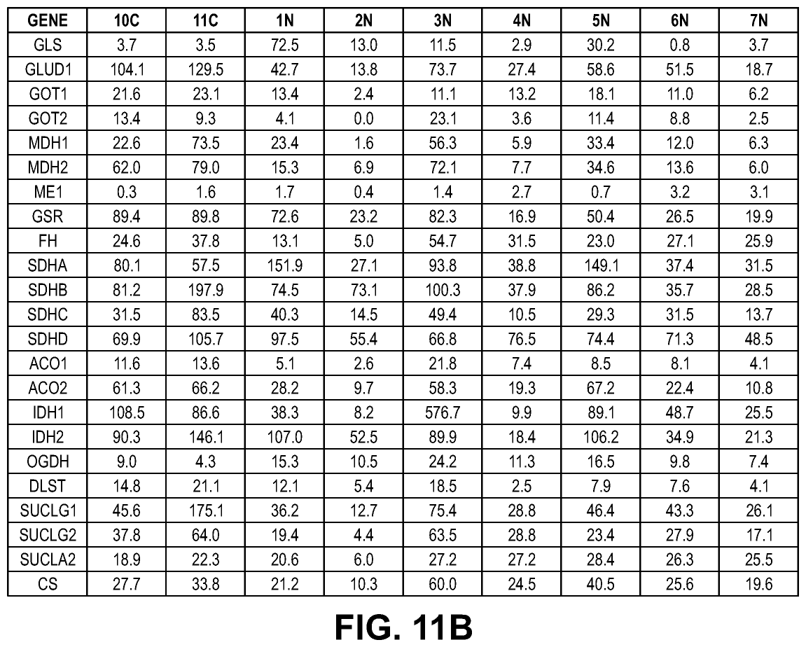

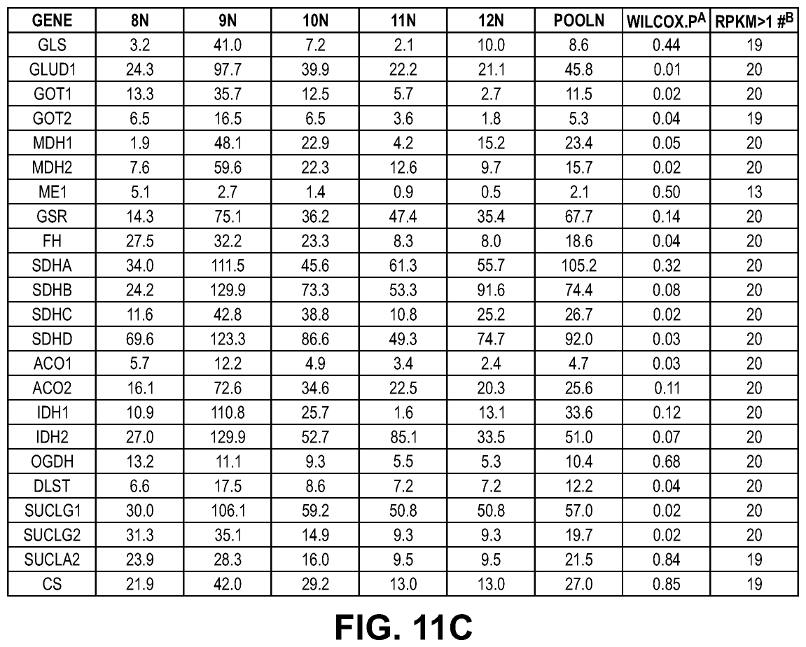

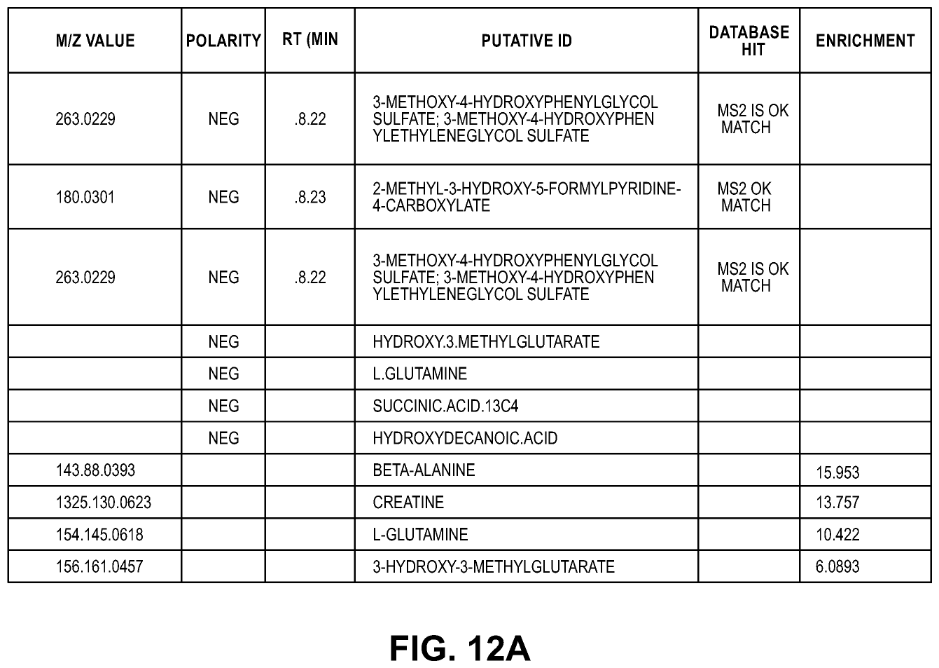

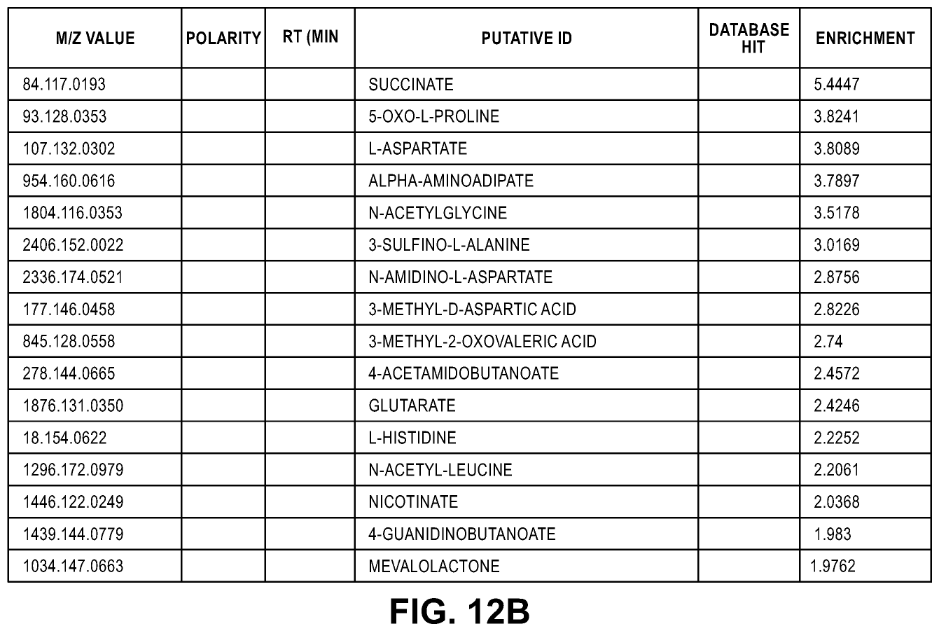

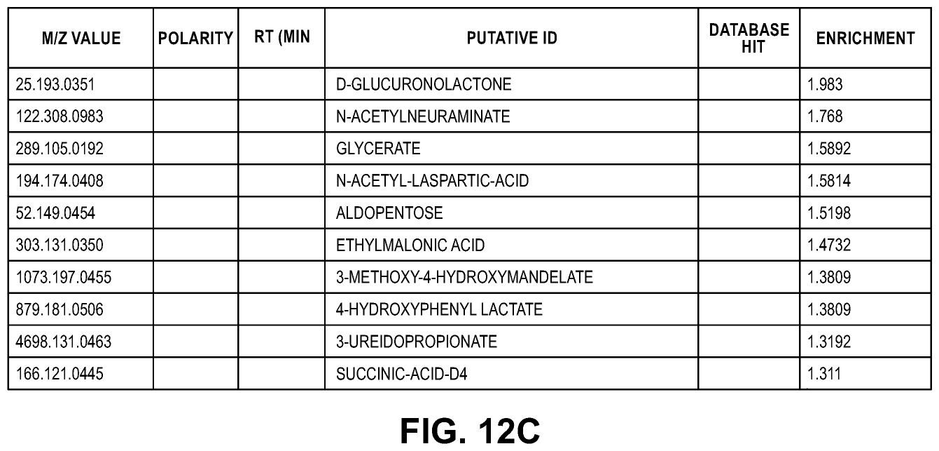

FIG. 10. NAD/NADH ratios in LNCaP and PC3 prostate cancer cell lines. The data from two independent experiments were expressed as mean±SD.

### Results

Deep Sequencing of Urine-Secreted mRNAs

Normal voided urine from men contains small numbers of exfoliated cells from different parts of the urinary tract including urothelial cells, squamous cells, renal tubular cells, and glandular cells including prostate epithelial cells. PCa cells are shed into urine and can be successfully isolated, processed, and analyzed by various molecular techniques, thereby providing a rich source of potential biomarkers for prostate cancer diagnosis and prognostication. It was desired to exploit this readily accessible and copious substrate from PCa patients for biomarker discovery and, in turn, elucidating novel mechanistic aspects of PCa.

Quality output from current next-generation sequencing (NGS) technology depends on the availability of high-quality RNA. An initial challenge was that the quality and quantity of RNA extracted from the very small number of exfoliated cells in urine was poor²⁶ (FIG. 5). To overcome the problem, sequence-specific capture (Illumina TruSeq™ RNA Access) was performed with the urine samples to reduce ribosomal RNA and enrich for exonic RNA sequences. With this approach, 11 PCa (for clinical details, see Table 4), 12 normal, and one pooled set of three normal samples (combined due to individually low RNA yields) were successfully sequenced. The 3825 RNA transcripts that were detected in 20 samples readily but not perfectly segregated the samples into normal and PCa groups (FIG. 6). It was concluded that RNA expression analysis of urine liquid biopsies by itself was unlikely to reveal sensitive and specific PCa biomarkers.

TABLE 4
The clinicopathological details of patients with
prostate cancer providing urine for RNA-seq.

| Sample | | | Pathological | Gleason | Age at | | LN |
|---|---|---|---|---|---|---|---|
| ID | Gender | PSA | Stage | grade | diagnosis | Diagnosis | Positive |
| 1C | M | 2.7 | pT2a | 6 | 48 | Prostatic | Not |
| | | | | | | Adenocarcinoma | submitted |
| 2C | M | | | | | | |
| 3C | M | 5.5 | pT2c | 6 | 58 | Prostatic | Negative for |
| | | | | | | Adenocarcinoma | tumor |
| 4C | M | 4.7 | pT2c | 7 | 69 | Prostatic | Negative for |
| | | | | | | Adenocarcinoma | tumor |
| 5C | M | 5.2 | pT3a | 7 | 69 | Prostatic | Not |
| | | | | | | Adenocarcinoma | submitted |
| 6C | M | 3.5 | pT2c | N/A | | Prostatic | Negative for |
| | | | | | | Adenocarcinoma | tumor |
| 7C | M | 2.2 | pT3a | 8 | 67 | Prostatic | Metastatic |
| | | | | | | Adenocarcinoma | |
| 9C | M | 11.8 | pT3a | 7 | 73 | Prostatic | Not |
| | | | | | | Adenocarcinoma | submitted |
| 8C | M | 7.9 | pT2c | 6 | 61 | Prostatic | Not |
| | | | | | | Adenocarcinoma | submitted |
| 10C | M | 4.2 | pT2a | 6 | 57 | Prostatic | Negative for |
| | | | | | | Adenocarcinoma | tumor |
| 11C | M | 4.6 | pT2c | 7 | 71 | Prostatic | Not |
| | | | | | | Adenocarcinoma | submitted |

M: male,
PSA: prostate antigen specific level (ng/ml),
LN: Lymph node,
N/A: not available

Cancer-specific gene signatures were next identified. Among 5510 differentially expressed transcripts, 4662 had reads per kilobase of transcript per million mapped reads (RPKM) values greater than one and 116 transcripts (111 genes) were significantly up- or downregulated in PCa (Tables 2 and 3). Differentially expressed genes were enriched for a number of important cancer pathways including PCa signaling, molecular mechanisms of cancer, PI3K/AKT signaling, and NF-κB signaling (Table 5). To the inventors knowledge, this is the first time that RNA-seq has been successfully applied to urine samples to profile coding genes.

TABLE 5
Pathways represented by genes differentially expressed in the cells
extracted from the urine of normal and cancer patients.

| Ingenuity Canonical | -Log (p- | |
|---|---|---|
| Pathways | value) | Genes |
| Molecular Mechanisms of | 10.5 | RAF1, GNAS, SMAD3, PIK3R1, CREBBP, TYK2, GNAQ, NCSTN, MDM2, |
| Cancer | | JAK2, XIAP, EP300, PTK2, SMAD4, CDKN1B, NOTCH1, BIRC3 |
| Pancreatic | 7.61 | RAF1, PIK3R1, SMAD3, TYK2, SMAD4, MDM2, JAK2, CDKN1B, NOTCH1 |
| Adenocarcinoma Signaling | | |
| PPARα/RXRα Activation | 7.42 | RAF1, GNAS, HSP90AB1, PLCG2, SMAD3, CREBBP, GNAQ, SMAD4, |
| | | JAK2, EP300 |
| Chronic Myeloid | 6.88 | HDAC6, RAF1, HDAC4, PIK3R1, SMAD3, SMAD4, MDM2, CDKN1B |
| Leukemia Signaling | | |
| IL-15 Production | 6.39 | PTK2, PTK2B, TYK2, JAK2, IRF1 |
| Renin-Angiotensin | 6.39 | PTK2, RAF1, PTK2B, PLCG2, PIK3R1, GNAQ, JAK2, ACE |
| Signaling | | |
| Prolactin Signaling | 6.32 | RAF1, PLCG2, PIK3R1, CREBBP, JAK2, IRF1, EP300 |
| PI3K/AKT Signaling | 6.31 | RAF1, HSP90AB1, PIK3R1, TYK2, MDM2, JAK2, CDKN1B, MCL1 |
| NF-κB Signaling | 6.1 | RAF1, MYD88, BCL10, PLCG2, PIK3R1, CREBBP, IGF1R, TNFAIP3, |
| | | EP300 |
| Prostate Cancer Signaling | 6.02 | RAF1, HSP90AB1, PIK3R1, CREBBP, MDM2, CDKN1B, EP300 |
| Hereditary Breast Cancer | 5.88 | HDAC6, NPM1, HDAC4, PIK3R1, SMARCB1, CREBBP, DDB2, EP300 |
| Signaling | | |
| Role of NFAT in Cardiac | 5.86 | HDAC6, RAF1, GNAS, HDAC4, PLCG2, PIK3R1, IGF1R, GNAQ, EP300 |
| Hypertrophy | | |
| Protein Kinase A | 5.82 | PTK2, RAF1, GNAS, PTPN2, ATF1, PTK2B, PLCG2 ,SMAD3, CREBBP, |
| Signaling | | GNAQ, SMAD4, EP300 |
| Cell Cycle: G1/S | 5.8 | HDAC6, HDAC4, SMAD3, SMAD4, MDM2, CDKN1B |
| Checkpoint Regulation | | |
| ERK/MAPK Signaling | 5.69 | PTK2, RAF1, ELF4, ATF1, PTK2B, PLCG2, PIK3R1, CREBBP, EP300 |
| Mouse Embryonic Stem | 5.63 | RAF1, PIK3R1, TYK2, CREBBP, SMAD4, JAK2, XIAP |
| Cell Pluripotency | | |
| Telomerase Signaling | 5.55 | HDAC6, TERF2, RAF1, ELF4, HDAC4, HSP90AB1, PIK3R1 |
| iNOS Signaling | 5.35 | MYD88, TYK2, CREBBP, JAK2, IRF1 |
| Glucocorticoid Receptor | 5.28 | RAFL1, ICAM1, HSP90AB1, PIK3R1, SMAD3, SMARCB1, CREBBP, |
| Signaling | | SMAD4, JAK2, EP300 |
| Tec Kinase Signaling | 5.26 | PTK2, GNAS, PTK2B, PLCG2, PIK3R1, TYK2, GNAQ, JAK2 |

Thirty-seven genes were significantly upregulated in PCa urine samples (Tables 2 and 3 and FIG. 1A) compared to normal urine samples. To bolster confidence that the RNA originated from the patient's cancer rather than contaminating cells, their expression in The Cancer Genome Atlas (TCGA) data (FIG. 1B and Table 6) was examined. Of these 37 genes, 35% (13/37) were significantly upregulated in primary tumors compared to normal (FIGS. 1B and 1C). Three of these genes were transcription factors (ELK4, SMARCB1, BRD3) and six were known oncogenes (TFG, NACA, BRD3, ELK4, NPM1, RPL22). When quantified in two representative PCa cell lines (LNCaP and PC3), most transcripts were upregulated in both cell lines compared to normal prostate epithelial cells (PrEC) except for NACA (downregulated in both cell lines), BRD3 and EPCAM (decreased in PC3 cells), and HDAC6 (downregulated in LNCaP cells) (FIG. 1D). Taken together, this data suggest that the transcriptional profiles generated from cells residing in urine from prostate cancer patients are likely to originate from cancerous prostate epithelial cells rather than other cellular contaminants from the urinary tract.

TABLE 6
A comparison of expression changes of genes differentially expressed in the urine of
patients with prostate cancer compared to normal in The Cancer Genome Atlas data.

| | Normal | | Tumor | | Δ | | |
|---|---|---|---|---|---|---|---|
| | Mean log2 | | Mean log2 | | mean log2 | | Up/ |
| Gene | (RSEM + 1) | SD | (RSEM + 1) | SD | (RSEM + 1) | p-value | downregulated |
| ACE | 10.2 | 1.2 | 9.1 | 0.9 | −1.1 | <0.0001 | Down |
| ATF1 | 8.9 | 0.3 | 8.7 | 0.5 | −0.2 | 0.0011 | Down |
| BRD3 | 10.4 | 0.3 | 10.5 | 0.5 | 0.2 | 0.0094 | Up |
| CCNB1IP1 | 9.3 | 0.4 | 9.1 | 0.5 | −0.2 | 0.0084 | Down |
| CDC14A | 8.5 | 0.9 | 8.7 | 0.9 | 0.2 | 0.0780 | |
| CDK8 | 7.6 | 0.4 | 7.1 | 0.7 | −0.5 | 0.0000 | Down |
| ELK4 | 8.2 | 0.9 | 8.5 | 0.8 | 0.2 | 0.0471 | Up |
| EPCAM | 11.3 | 0.8 | 12.5 | 0.7 | 1.2 | <0.0001 | Up |
| FH | 10.2 | 0.3 | 10.4 | 0.3 | 0.2 | 0.0006 | Up |
| GMPS | 9.6 | 0.3 | 9.6 | 0.4 | 0.1 | 0.2340 | |
| GNAS | 14.3 | 0.3 | 14.3 | 0.4 | 0.0 | 0.5377 | |
| GOT1 | 10.0 | 0.6 | 9.8 | 0.4 | −0.2 | 0.0025 | Down |
| GRHPR | 10.9 | 0.3 | 11.2 | 0.3 | 0.3 | <0.0001 | Up |
| HDAC6 | 9.9 | 0.4 | 10.0 | 0.3 | 0.1 | 0.0358 | Up |
| HSP90AB1 | 14.7 | 0.3 | 14.9 | 0.7 | 0.2 | 0.0659 | |
| LRPPRC | 11.4 | 0.2 | 11.5 | 0.4 | 0.1 | 0.0944 | |
| MSH3 | 8.9 | 0.3 | 8.8 | 0.5 | −0.1 | 0.3042 | |
| NACA | 13.8 | 0.4 | 14.2 | 0.5 | 0.4 | <0.0001 | Up |
| NPM1 | 12.8 | 0.4 | 13.3 | 0.4 | 0.5 | <0.0001 | Up |
| PFDN5 | 12.3 | 0.5 | 12.3 | 0.6 | 0.0 | 0.7540 | |
| PHB | 11.5 | 0.3 | 11.6 | 0.4 | 0.1 | 0.0224 | Up |
| PHF6 | 9.3 | 0.5 | 9.3 | 0.6 | −0.1 | 0.3891 | |
| PIK3R1 | 11.4 | 0.6 | 10.3 | 0.7 | −1.1 | <0.0001 | Down |
| PTK2 | 11.1 | 0.2 | 11.0 | 0.4 | −0.1 | 0.1724 | |
| PTPN2 | 8.8 | 0.3 | 8.8 | 0.3 | 0.0 | 0.4067 | |
| RPL22 | 12.9 | 0.3 | 13.2 | 0.4 | 0.3 | <0.0001 | Up |
| RPS11 | 14.6 | 0.5 | 15.0 | 0.6 | 0.4 | <0.0001 | Up |
| SDHC | 10.8 | 0.2 | 10.8 | 0.3 | 0.0 | 0.4445 | |
| SDHD | 11.0 | 0.3 | 10.4 | 0.4 | −0.6 | <0.0001 | Down |
| SMAD4 | 11.4 | 0.3 | 11.1 | 0.4 | −0.3 | <0.0001 | Down |
| SMARCB1 | 10.6 | 0.3 | 10.7 | 0.3 | 0.1 | 0.0059 | Up |
| TCEA1 | 10.6 | 0.3 | 10.6 | 0.4 | 0.0 | 0.6120 | |
| TERF2 | 9.2 | 0.2 | 9.2 | 0.4 | −0.1 | 0.1909 | |
| TFG | 10.9 | 0.5 | 11.0 | 0.3 | 0.1 | 0.0114 | Up |
| TMEM230 | 11.6 | 0.4 | 11.6 | 0.3 | −0.1 | 0.1750 | |
| ZMYND11 | 11.6 | 0.3 | 11.1 | 0.4 | −0.4 | <0.0001 | Down |
| ZNF585B | 6.4 | 0.5 | 5.9 | 0.8 | −0.5 | <0.0001 | Down |

Metabolomic Profiling of Urine from Normal Subjects and Patients with Diseased Prostates

Targeted or global strategies have been used to profile metabolites in urine samples and identify PCa biomarkers, but results have been highly variable. In the first unbiased metabolomics study which measured 1126 metabolites in 262 clinical samples including 110 urine samples, the glycine derivative sarcosine was elevated in PCa tissue and urine from PCa patients, and functional validation of the oncogenic role of sarcosine was provided in vitro. However, sarcosine was not a reproducible prognostic marker in independent cohorts, a common finding in single-biomarker studies that possess neither the specificity nor sensitivity for clinical development.

With this in mind, both global and targeted metabolite profiling was performed of urine from patients with normal prostates, BPH, prostatitis, and PCa to discover cancer-specific metabolic changes. In global metabolite profiling, the metabolic profiles of urine specimens from normal subjects and patients with cancer were distinct and separate using principal component analysis (PCA), whilst there was significant overlap between the profiles obtained from patients with BPH and prostatitis (FIG. 2). Positive and negative ion data were first normalized to the specific gravity and then normalized to the total ion signal for all subsequent statistical analyses. Positive and negative ion data sets were treated separately and initial analysis was performed with PCA. The negative ion data set separation by PCA (FIG. 2A) was very distinct between PCa and the control group with BPH and prostatitis clustering together but as a separate cluster from PCa and control. Separation was primarily observed along PC1. However, no correlation was observed between PSA scores and metabolic profiling. A heat map using only the top 25 metabolites from ANOVA showed excellent clustering of the PCa and normal group and no discrimination of the prostatitis and BPH groups (FIG. 2B), highlighting the promise of metabolomics for identifying new and unique disease-associated metabolites. The unknowns were next removed and known metabolite expression was focused on. ANOVA showed 22 significant metabolites (FDR corrected p-value <0.05) in the negative ion data set. The most significant metabolite difference was a peak corresponding to Ala or sarcosine, which discriminated all other groups and cancer (p=1.9×10⁻¹⁵) but was not significantly different in BPH vs. cancer vs. prostatitis. This was also the case for creatine (p=5.5×10⁻¹⁴), while Gln was significantly different between all groups (FIGS. 2C and D).

In targeted metabolic profiling, amino acids (23 species), acylcarnitines (24 species), and organic acids (8 species) were measured in urine samples. Prostatitis, BPH, and PCa are all associated with inflammatory infiltrates in the majority of cases that participate in cancer progression, but the separation of PCa cases as a distinct subgroup suggests distinct metabolic abnormalities in PCa arising either systemically or in the cancer itself.

Overall, 14/23 (61%) amino acids showed significant up- or downregulation between groups (Table 7 and FIG. 7). Compared to normal, six amino acids (1-methylhistidine, Asp, Glu, Ile, ornithine, and Pro) were significantly upregulated in BPH, prostatitis, or PCa urine samples: Glu was highly and significantly increased in prostatitis (699% increase) and PCa (1450% increase), and Asp (631% increase) and Glu (1561% increase) were upregulated in PCa compared to BPH. Eight amino acids were significantly decreased in PCa urine samples compared to normal urine samples (by between 42% and 65%). 4/8 organic acids showed significant changes compared to normal urine samples (Table 7 and FIG. 7). None were increased in PCa urine samples, but two (pyruvate, 63%; α-ketoglutarate, 69%) were decreased in PCa samples. Succinate was increased in BPH and prostatitis urine samples (115% and 393%, respectively). Eleven out of twenty four acylcarnitines showed significant changes between groups (Table 7 and FIG. 7): C4-OH butyryl, C4-OH isobutyryl, and C10 were increased in PCa urine samples compared to normal and prostatitis, while C6 and C6-OH were increased in PCa compared to prostatitis urine samples. Interestingly, five acylcarnitine species (C3, C4 isobutyryl, C4-methylmanlonyl, C5 2-methylbutyryl, and C5 isovaleryl) were decreased in PCa compared to BPH.

TABLE 7
Metabolites showing significantly different levels between clinical groups.

| | Prostatitis vs. | | BPH vs. | | Prostatitis vs. | | PCa vs. | | PCa vs. | | PCa vs. | |
|---|---|---|---|---|---|---|---|---|---|---|---|---|
| | BPH | | Normal | | Normal | | BPH | | Prostatitis | | Normal | |
| | % | P | % | P | % | P | % | P | % | P | % | P |
| | change | value | change | value | change | value | change | value | change | value | change | value |
| Amino Acids | | | | | | | | | | | | |
| 1- | | | 35.2 | 0.002 | 25.4 | 0.031 | | | | | | |
| Methylhistidine | | | | | | | | | | | | |
| Alanine | | | | | | | −49.1 | 0.013 | | | −46.9 | 0.008 |
| Aspartate | 242.7 | 0.001 | −25.7 | 0.021 | 154.8 | 0.006 | 631.0 | 0.012 | | | 443.4 | 0.021 |
| Citrulline | | | | | | | −54.4 | 0.005 | | | −43.2 | 0.015 |
| Glutamate | | | | | 698.6 | 0.000 | 1561.4 | 0.010 | | | 1450.1 | 0.012 |
| Glutamine | | | | | −43.4 | 0.000 | −68.0 | 0.013 | | | −65.0 | <0.001 |
| Histidine | | | | | | | −44.3 | 0.003 | −42.3 | 0.012 | −54.9 | <0.001 |
| Isoleucine | | | | | 25.6 | 0.041 | | | | | | |
| Lysine | | | | | | | −58.4 | 0.011 | | | −50.1 | 0.041 |
| Methionine | | | | | −30.9 | 0.020 | −47.1 | 0.009 | | | −42.5 | 0.001 |
| Ornithine | | | 54.4 | 0.011 | | | −32.7 | 0.044 | | | | |
| Proline | | | | | 59.8 | 0.000 | | | | | 121.1 | 0.005 |
| Threonine | | | | | | | −36.6 | 0.039 | | | −42.3 | 0.009 |
| Tyrosine | | | | | | | −38.5 | 0.026 | | | −43.8 | 0.007 |
| Organic Acids | | | | | | | | | | | | |
| Pyruvate | | | −40.3 | 0.030 | −74.3 | <0.001 | | | | | −63.1 | 0.001 |
| Succinate | 128.9 | 0.002 | 115.3 | 0.004 | 392.8 | <0.001 | | | | | | |
| Malate | | | 65.8 | 0.046 | | | | | | | | |
| α-ketoglutarate | −47.9 | 0.029 | −57.3 | <0.001 | −77.7 | <0.001 | | | | | −69.3 | <0.001 |
| Acylcarnitine | | | | | | | | | | | | |
| C3 | | | | | | | −75.2 | 0.005 | | | −66.2 | 0.044 |
| C3-DC | −22.3 | 0.025 | | | | | | | | | | |
| C4 isobutyryl | | | | | | | −56.2 | 0.012 | | | | |
| C4- | −29.0 | 0.005 | 22.9 | 0.016 | | | −26.9 | 0.007 | | | | |
| methylmanlonyl | | | | | | | | | | | | |
| C4-OH butyryl | | | | | | | | | 692.3 | 0.035 | 309.7 | 0.022 |
| C4-OH | | | | | | | | | 131.8 | 0.010 | 77.9 | 0.009 |
| isobutyryl | | | | | | | | | | | | |
| C5 2- | | | | | | | −43.8 | 0.039 | | | | |
| methylbutyryl | | | | | | | | | | | | |
| C5 isovaleryl | | | | | | | −70.5 | 0.024 | | | | |
| C6 | | | | | | | | | 229.5 | 0.017 | | |
| C6-OH | −67.8 | 0.004 | | | | | | | 244.8 | 0.021 | | |
| C10 | | | | | | | 123.6 | 0.010 | 189.0 | 0.016 | 126.7 | 0.009 |

Prostate carcinogenesis is known to involve metabolic reprogramming to provide sufficient energy for rapid cellular proliferation. Many cancer cells exhibit augmented aerobic glycolysis, known as the Warburg effect, even in high-oxygen environments. This metabolic adaptation helps provide essential cellular components such as lipids and nucleotides to support the anabolic needs of rapidly proliferating tumor cells. Beyond the Warburg effect, the TCA cycle and oxidative phosphorylation also play important roles in PCa. Prostate epithelial cells normally produce certain components of prostatic fluid such as citrate, PSA, and polyamines. Increased citrate production by prostate cells means that they favor citrate synthesis over citrate utilization. However, PCa cells degrade citrate and accumulate oxidized citrate, resulting in more efficient energy production.

MetaboAnalyst™ was next used to identify the molecular pathways most strongly linked to the altered metabolite profiles. These deduced pathways included Ala, Asp, and Glu metabolism; D-Gln and D-Glu metabolism; and Arg and Pro metabolism, with Ala, Asp, and Glu metabolism expected to have the most impact (FIG. 3A and Table 8). The acylcarnitine profiles were not impactful in the MetaboAnalyst™ analysis due to a relative paucity of KEGG IDs for the measured set of acylcarnitines. Nevertheless, the accumulation of urinary acylcarnitines in PCa is consistent with recent findings of increased circulating acylcarnitines in prostate cancer patients and abnormal fatty acid utilization for energy production.

TABLE 8
The top ten pathways represented by differential levels in normal
and diseased urine (MetaboAnalyst ™ 3.0).

| | Match | | | | | |
|---|---|---|---|---|---|---|
| Pathway Name | no. | p-value | −log(p) | Holm p | FDR | Impact |
| Aminoacyl-tRNA | 10/75 | 3.2367E−12 | 26.456 | 2.5894E−10 | 2.5894E−10 | 0.11268 |
| biosynthesis | | | | | | |
| Alanine, aspartate | 5/24 | 2.5745E−7 | 15.172 | 2.0339E−5 | 1.0298E−5 | 0.52897 |
| and glutamate | | | | | | |
| metabolism | | | | | | |
| Arginine and | 6/77 | 5.473E−6 | 12.116 | 4.2689E−4 | 1.4595E−4 | 0.25184 |
| proline metabolism | | | | | | |
| Nitrogen | 4/39 | 9.3233E−5 | 9.2804 | 0.007179 | 0.0018647 | 6.7E−4 |
| metabolism | | | | | | |
| Cysteine and | 4/56 | 3.8874E−4 | 7.8526 | 0.029544 | 0.0062199 | 0.05455 |
| methionine | | | | | | |
| metabolism | | | | | | |
| Valine, leucine | 3/27 | 6.4002E−4 | 7.354 | 0.048001 | 0.0085336 | 0.03498 |
| and isoleucine | | | | | | |
| biosynthesis | | | | | | |
| D-Glutamine and | 2/11 | 0.002201 | 6.1189 | 0.16287 | 0.025154 | 0.35294 |
| D-glutamate | | | | | | |
| metabolism | | | | | | |
| Histidine | 3/44 | 0.0027041 | 5.913 | 0.1974 | 0.025649 | 0.14548 |
| metabolism | | | | | | |
| Phenylalanine | 3/45 | 0.0028855 | 5.8481 | 0.20776 | 0.025649 | 0.0 |
| metabolism | | | | | | |
| Glycine, serine | 3/48 | 0.0034744 | 5.6623 | 0.24668 | 0.027795 | 0.09661 |
| and threonine | | | | | | |
| metabolism | | | | | | |

Integrated Gene Expression and Metabolite Analysis

It was reasoned that integrating changes in gene expression and metabolite levels evident in the urine samples would better unveil the key pathways driving the biological processes in PCa and hence pinpoint the most robust biomarkers. The integrated pathway analysis module of MetaboAnalyst™ was used to map both genes and metabolites to KEGG pathways to determine not just overrepresented pathways but the relative importance of the genes and compounds based on their relative locations (topology). The top three pathways most significantly enriched for differentially expressed genes and metabolites were: aminoacyl-tRNA biosynthesis; Ala, Asp, and Glu metabolism; and the TCA cycle) (p<0.001; FIG. 3B and Table 9). Aminoacyl-tRNA biosynthesis probably represents an increase in global protein translation and demand for protein synthesis in cancer cells. However, Ala, Asp, and Glu metabolism and the TCA cycle are closely related pathways that are critical for energy generation and carbon and nitrogen metabolism for biomass accumulation, especially in rapidly dividing cells such as cancer cells.

TABLE 9
The top ten pathways most significantly enriched for differentially
expressed genes and metabolites using an integrated analysis in MetaboAnalyst ™ 3.0.

| Pathway | Total | Expected | Hits | p-value | Topology |
|---|---|---|---|---|---|
| Aminoacyl-tRNA | 87 | 0.85134 | 10 | 2.912E−9 | 0.14493 |
| biosynthesis | | | | | |
| Alanine, aspartate and | 56 | 0.54799 | 6 | 1.1105E−5 | 0.55102 |
| glutamate metabolism | | | | | |
| Citrate cycle (TCA cycle) | 50 | 0.48927 | 5 | 9.4033E−5 | 0.36364 |
| Arginine and proline | 102 | 0.99812 | 6 | 3.4034E−4 | 0.21505 |
| metabolism | | | | | |
| D-Glutamine and D- | 9 | 0.088069 | 2 | 0.0031787 | 0.28571 |
| glutamate metabolism | | | | | |
| Phenylalanine, tyrosine and | 9 | 0.088069 | 2 | 0.0031787 | 1.4 |
| typtophan biosynthesis | | | | | |
| Valine, leucine and | 13 | 0.12721 | 2 | 0.0067233 | 0.18182 |
| isoleucine biosynthesis | | | | | |
| Histidine metabolism | 44 | 0.43056 | 3 | 0.0084469 | 0.3125 |
| Cysteine and methionine | 63 | 0.61648 | 3 | 0.022399 | 0.32727 |
| metabolism | | | | | |
| Phenylalanine metabolism | 29 | 0.28378 | 2 | 0.031798 | 0.22727 |

Glutamate Metabolism Contributes to the Cancerous Phenotype Via GOT1-Mediated Redox Balance

GOT1, a cytosolic transaminase that converts Asp to Glu, and other genes involved in Gln metabolism such as GLUD1, GOT1, GOT2, and MDH1 were significantly upregulated in PCa urine samples (FIG. 8 and Table 10). To investigate the role of GOT1 as a regulatory metabolic node in prostate cancer, GOT1 was knocked down in the prostate cancer cell lines LNCaP and PC3 using siRNA (FIG. 4A). As expected, GOT1 knockdown upregulated the upstream metabolites (FIGS. 8 and 9), Glu [1.2-fold (LNCaP; p=0.01) and 1.4-fold (PC3; p=0.03)] and Asp (1.5-fold (LNCaP; p=0.0004) and 2.6-fold (PC3; p=0.0006)], in both cells lines. GOT1 knockdown significantly decreased the viability of both LNCaP and PC3 cells (FIG. 4B), consistent with previous reports that GOT1 repression suppresses tumor growth, and the colony forming ability and invasiveness of PC3 cells (FIGS. 4C and D). The mechanism by which GOT1 regulated prostate cancer cell viability was therefore examined.

Maintaining NAD/NADH balance supports de novo Asp biosynthesis and is required for proliferation. Since GOT1 is part of the malate-Asp shuttle, whether GOT1 knockdown could affect the NAD/NADH ratio was checked. The results illustrated that NAD/NADH ratio was indeed decreased (FIG. 10) suggesting that this reduction may have influenced the cell proliferation inhibition in both LNCaP and PC3 cells. As depicted in FIG. 8, GOT1 is necessary to convert Asp derived from the Gln TCA cycle into oxaloacetate and malate to produce NADPH, which is essential for maintaining intracellular redox balance via detoxification of damaging reactive oxygen species (ROS). Both LNCaP and PC3 cells showed increased ROS levels upon GOT1 knockdown (FIG. 4E), suggesting that GOT1 plays a role in cellular redox balance and can be manipulated to reduce the viability of prostate cancer cells.

### DISCUSSION

A group of putative RNA and metabolite biomarkers in urine was identified and unveiled a novel therapeutic target for prostate cancer. To improve the accuracy of disease classification, metabolic and transcriptomic profiling of urines from BPH, prostatitis, and PCa patients (without prostatic massage) was carried out. Urine from normal healthy individuals was used as the control comparator. Through an integrated analysis of metabolomic and transcriptomic data, GOT1 was identified as a key regulator of metabolic changes in PCa patients.

Recent advances in transcriptomics and metabolomics have led to the identification of various candidate biomarkers for cancer diagnosis and prognosis. However, biomarkers derived from one dataset may not be reliable and reproducibility in independent cohorts is challenging. There are great advantages to using biofluids including blood, urine, saliva, and seminal plasma as sources of biomarkers. Among them, urine is a promising source of liquid biopsy as it is noninvasive, replenishable, and convenient to collect. In addition to proteins and peptides, urine contains various nucleic acids, metabolites, and lipids. Recently, the long noncoding RNA PCA3 and the fusion gene TMPRSS2:ERG have been proposed as urinary PCa biomarkers. Here, disclosed for the first time is a global transcriptomic profile of PCa in urine. Capture-based enrichment (RNA Access protocol) was applied, in which probes target exonic regions, and were able to separate PCa samples from normal healthy individual samples by unsupervised methods. Here it is shown that beta-alanine/sarcosine was enriched in BPH, prostatitis, and PCa urine samples.

In pancreatic ductal adenocarcinoma (PDAC), the transaminase GOT1, is required to sustain cell growth by enabling the production of NADPH to compensate internal ROS. In this study, it was determined that GOT1 is essential for prostate cancer cell line (PC3 and LNCaP) growth. GOT1 knockdown increased ROS levels, suggesting that GOT1 may be involved in NADPH generation. GOT1 also functions as a member of the malate-aspartate shuttle, in which two pairs of enzymes, glutamate oxaloacetate transaminases (GOT) and malate dehydrogenase (MDH) serve to transfer reducing equivalents across the mitochondrial membrane. The transcriptomic analysis described herein revealed the upregulation of all members of the shuttle including GOT1, GOT2, MDH1, and MDH2. These results suggest that the malate-aspartate shuttle may play an important role in cell growth in PCa. This hypothesis is supported by the reduction in NAD/NADH ratio upon GOT1 knockdown in both cell lines.

In conclusion, prostate cancers appear to undergo GOT1-dependent metabolic adaptation to promote a malignant phenotype and resist oxidative stress. The glutamate phenotype represented by the gene expression and metabolic changes in urine reflect this GOT1-dependent pathway in prostate cancer cells. In addition to focusing on these pathway components as biomarkers of prostate cancer in urine, enzymes involved in this pathway might be excellent targets for PCa therapy. Indeed, small molecule inhibitors of GLS1 (mitochondrial glutaminase), which converts glutamine to glutamate, already exist. Targeting this pathway is worthy of further investigation either with or without concurrent ROS-induced cellular stress, this latter approach a particularly appealing strategy in patients with prostate cancers treated with ionizing radiotherapy. Liquid biopsies are an extremely useful tool for non-invasive biomarker and target discovery.

Although the invention has been described with reference to the above examples, it will be understood that modifications and variations are encompassed within the spirit and scope of the invention. Accordingly, the invention is limited only by the following claims.

What is claimed is:

1. A method comprising:
a) detecting an expression level of a gene in a liquid sample from a subject having or suspected of having prostate cancer, wherein the gene is selected from one or more genes recited in Table 2, Table 3 and/or Table 6; and/or
b) detecting a level of a metabolite in the sample, wherein the metabolite comprises one or more amino acids, organic acids or acylcarnitine derivatives as set forth in Table 7 and/or Table 11.

2. The method of claim 1, further comprising detecting the expression level of the gene and detecting the level of the metabolite, wherein the metabolic pathway is selected from aminoacyl-tRNA biosynthesis, Ala, Asp, and Glu metabolism, or the TCA cycle.

3. The method of claim 1, wherein the sample is a bodily fluid such as serum, plasma, feces, or urine.

4. The method of claim 3, wherein the sample is urine.

5. The method of claim 1, wherein the subject is a mammal.

6. The method of claim 5, wherein the mammal is a human.

7. The method of claim 1, wherein the metabolite is one or more amino acids selected from the group consisting of 1-Methylhistidine, Alanine, Aspartate, Citrulline, Glutamate, Glutamine, Histidine, Isoleucine, Lysine, Methionine, Ornithine, Proline, Threonine, Tyrosine, L-Aspartate, L-Proline, N-Acetylglycine, 5-oxo-L-Proline, 3-sulfino-L-Alanine, beta-Alanine, N-methyl-D-Aspartate, N-Acetyl-L-Leucine, creatine, and combinations thereof.

8. The method of claim 7, wherein the one or more amino acids consists essentially of 1-Methylhistidine, Aspartate, Glutamate, Isoleucine, Ornithine and Proline.

9. The method of claim 7, wherein the one or more amino acids consists essentially of Alanine, Aspartate, Glutamate or combination thereof.

10. The method of claim 1, wherein the metabolite is one or more organic acids selected from the group consisting of Pyruvate, Succinate, Malate and α-ketoglutarate, 3-hydroxy-3-Methylglutarate, 4-Guanidinobutanoate, D-Glucuronolactone and combinations thereof.

11. The method of claim 1, wherein the metabolite is one or more acylcarnitine derivatives selected from the group consisting of C3, C3-DC, C4 isobutyryl, C4-methylmanlonyl, C4-OH butyryl, C4-OH isobutyryl, C5 2-methylbutyryl, C5 isovaleryl, C6, C6-OH and C10.

12. The method of claim 1, wherein the one or more genes is selected from the group consisting of EPCAM, GRHPR, HDAC6, PHB, RPS11, GOT1, ELK4, SMARCB1, BRD3, TFG, NACA, NPM1, RPL22, and combinations thereof.

13. The method of claim 1, wherein detecting the expression level comprises measuring an expression product.

14. The method of claim 13, wherein the expression product is protein, microRNA or mRNA.

15. The method of claim 1, wherein detecting the expression level comprises measuring RNA, and wherein the sample is urine.

16. The method of claim 1, wherein the method further comprises histological analysis of a biopsy tissue.

17. The method of claim 1, further comprising administering a therapeutic agent to the subject.

18. The method of claim 1, further comprising prescribing the subject a therapeutic regime.

19. A method of determining a treatment for prostate cancer in a subject having or suspected of having prostate cancer, comprising:
a) detecting an expression level of a gene in a sample from a subject having or suspected of having prostate cancer, wherein the gene is selected from one or more genes recited in Table 2, Table 3 and/or Table 6; and/or detecting a level of a metabolite in the sample, wherein the metabolite comprises one or more amino acids, organic acids or acylcarnitine derivatives as set forth in Table 7 and/or Table 11; and
b) administering a treatment to the subject.

20. The method of claim 19, further comprising detecting the expression level of the gene and detecting the level of the metabolite.

21. The method of claim 19, wherein the sample is a bodily fluid such as serum, plasma, feces, or urine.

22. The method of claim 21, wherein the sample is urine.

23. The method of claim 19, wherein the subject is a mammal.

24. The method of claim 23, wherein the mammal is a human.

25. The method of claim 19, wherein the metabolite is one or more amino acids selected from the group consisting of 1-Methylhistidine, Alanine, Aspartate, Citrulline, Glutamate, Glutamine, Histidine, Isoleucine, Lysine, Methionine, Ornithine, Proline, Threonine, Tyrosine, L-Aspartate, L-Proline, N-Acetylglycine, 5-oxo-L-Proline, 3-sulfino-L-Alanine, beta-Alanine, N-methyl-D-Aspartate, N-Acetyl-L-Leucine, creatine, and combinations thereof.

26. The method of claim 25, wherein the one or more amino acids consists essentially of 1-Methylhistidine, Aspartate, Glutamate, Isoleucine, Ornithine and Proline.

27. The method of claim 25, wherein the one or more amino acids consists essentially of Aspartate, Glutamate or combination thereof.

28. The method of claim 19, wherein the metabolite is one or more organic acids selected from the group consisting of Pyruvate, Succinate, Malate and α-ketoglutarate, 3-hydroxy-3-Methylglutarate, 4-Guanidinobutanoate, D-Glucuronolactone and combinations thereof.

29. The method of claim 19, wherein the metabolite is one or more acylcarnitine derivatives selected from the group consisting of C3, C3-DC, C4 isobutyryl, C4-methylmanlonyl, C4-OH butyryl, C4-OH isobutyryl, C5 2-methylbutyryl, C5 isovaleryl, C6, C6-OH and C10.

30. The method of claim 19, wherein the one or more genes is selected from the group consisting of EPCAM, GRHPR, HDAC6, PHB, RPS11, GOT1, ELK4, SMARCB1, BRD3, TFG, NACA, NPM1, and RPL22.

31. The method of claim 19, wherein detecting the expression level comprises measuring an expression product.

32. The method of claim 31, wherein the expression product is protein, microRNA or mRNA.

33. The method of claim 19, wherein detecting the expression level comprises measuring RNA, and wherein the sample is urine.

34. The method of claim 19, wherein the method further comprises histological analysis of a biopsy tissue.

35. The method of claim 19, wherein the treatment comprises administering a chemotherapeutic agent and/or treatment therapy.

36. A method of diagnosing cancer in a sample from a subject having or at risk of having prostate cancer comprising:
a) detecting an expression level of a gene in a sample from a subject having or suspected of having prostate cancer, wherein the gene is selected from one or more genes recited in Table 2, Table 3 or Table 6; and optionally, detecting a level of a metabolite in the sample, wherein the metabolite comprises one or more amino acids, organic acids or acylcarnitine derivatives as set forth in Table 7 and/or Table 11; and
b) diagnosing cancer in the subject, wherein the expression level, or level of the metabolite in the sample, being up-regulated or down-regulated as compared to a corresponding normal sample is indicative of prostate cancer, thereby diagnosing cancer in the subject.

37. The method of claim 36, further comprising detecting the expression level of the gene and detecting the level of the metabolite.

38. The method of claim 36, wherein the sample is a bodily fluid such as serum, plasma, feces, or urine.

39. The method of claim 38, wherein the sample is urine.

40. The method of claim 36, wherein the subject is a mammal.

41. The method of claim 40, wherein the mammal is a human.

42. The method of claim 36, wherein the metabolite is one or more amino acids selected from the group consisting of 1-Methylhistidine, Alanine, Aspartate, Citrulline, Glutamate, Glutamine, Histidine, Isoleucine, Lysine, Methionine, Ornithine, Proline, Threonine, Tyrosine, L-Aspartate, L-Proline, N-Acetylglycine, 5-oxo-L-Proline, 3-sulfino-L-Alanine, beta-Alanine, N-methyl-D-Aspartate, N-Acetyl-L-Leucine, creatine, and combinations thereof.

43. The method of claim 42, wherein the one or more amino acids consists essentially of 1-Methylhistidine, Aspartate, Glutamate, Isoleucine, Ornithine and Proline.

44. The method of claim 42, wherein the one or more amino acids consists essentially of Aspartate, Glutamate or combination thereof.

45. The method of claim 36, wherein the metabolite is one or more organic acids selected from the group consisting of Pyruvate, Succinate, Malate and α-ketoglutarate, 3-hydroxy-3-Methylglutarate, 4-Guanidinobutanoate, D-Glucuronolactone and combinations thereof.

46. The method of claim 36, wherein the metabolite is one or more acylcarnitine derivatives selected from the group consisting of C3, C3-DC, C4 isobutyryl, C4-methylmanlonyl, C4-OH butyryl, C4-OH isobutyryl, C5 2-methylbutyryl, C5 isovaleryl, C6, C6-OH and C10.

47. The method of claim 36, wherein the one or more genes is selected from the group consisting of EPCAM, GRHPR, HDAC6, PHB, RPS11, GOT1, ELK4, SMARCB1, BRD3, TFG, NACA, NPM1, and RPL22.

48. The method of claim 36, wherein detecting the expression level comprises measuring an expression product.

49. The method of claim 48, wherein the expression product is protein, microRNA or mRNA.

50. The method of claim 36, wherein detecting the expression level comprises measuring RNA, and wherein the sample is urine.

51. The method of claim 36, wherein the method further comprises histological analysis of a biopsy tissue.

52. The method of claim 36, further comprising administering a therapeutic agent to the subject.

53. The method of claim 36, further comprising prescribing the subject a therapeutic regime.

54. A probe set for detecting or assessing prostate cancer, comprising a plurality of probes, wherein each probe is capable of detecting an expression level of a gene selected from those recited in Table 2, Table 3 and/or Table 6.

55. The probe set of claim 54, wherein the plurality of probes are oligonucleotides.

56. An array comprising a plurality of probes for detecting the expression level of a gene selected from those recited in Table 2, Table 3 and/or Table 6.

57. The array of claim 56, wherein the plurality of probes are oligonucleotides.

58. The array of claim 56, wherein the plurality of probes are polypeptides.

59. The array of claim 56, wherein the plurality of probes are antibodies.

60. A kit comprising:
a) the probe set of claim 54 or an array of claim 56; and optionally,
b) reagents for detecting a level of a metabolite in a sample, wherein the metabolite comprises one or more amino acids, organic acids or acylcarnitine derivatives as set forth in Table 7.

61. A method of inhibiting the growth of prostate cancer cells comprising administering to the cell a GOT1 inhibitor, thereby reducing the growth of the prostate cancer cells.

62. The method of claim 61, wherein the inhibitor is a small molecule, a peptide, or a nucleic acid molecule.

63. The method of claim 61, wherein the inhibitor is an siRNA molecule.

64. The method of any of claim 1, 19 or 36, wherein the prostate cancer is PCa or BPH.


SEQUENCE LISTING (34 sequences)

SEQ ID NO: 1 - DNA, 20 bp, Homo sapiens
ctgaaacccaccactttgcg

SEQ ID NO: 2 - DNA, 20 bp, Homo sapiens
gcttgctgagaacggtttcc

SEQ ID NO: 3 - DNA, 20 bp, Homo sapiens
gccccttgctctccagtatc

SEQ ID NO: 4 - DNA, 20 bp, Homo sapiens
catccagcccagacagagtg

SEQ ID NO: 5 - DNA, 18 bp, Homo sapiens
gttcgggcttctgcttgc

SEQ ID NO: 6 - DNA, 20 bp, Homo sapiens
cagtttacggccagcttgta

SEQ ID NO: 7 - DNA, 18 bp, Homo sapiens
ccgagcacttcggctcct

SEQ ID NO: 8 - DNA, 22 bp, Homo sapiens
atccggaaggaattttggcttg

SEQ ID NO: 9 - DNA, 20 bp, Homo sapiens
gaccacgtggacaagaggat

SEQ ID NO: 10 - DNA, 20 bp, Homo sapiens
tctgtcaggacatctggggt

SEQ ID NO: 11 - DNA, 20 bp, Homo sapiens
gctgactacctagctgcctg

SEQ ID NO: 12 - DNA, 20 bp, Homo sapiens
tcaaagccagctgagaccag

SEQ ID NO: 13 - DNA, 20 bp, Homo sapiens
cacgctctcgctcggtcttt

SEQ ID NO: 14 - DNA, 22 bp, Homo sapiens
ggctgaagacataggtagcaca

SEQ ID NO: 15 - DNA, 20 bp, Homo sapiens
actccagccaaaaatgcaca

SEQ ID NO: 16 - DNA, 23 bp, Homo sapiens
tacatgtagtgcccaggactgtt

SEQ ID NO: 17 - DNA, 20 bp, Homo sapiens
atcacccagagagagctggt

SEQ ID NO: 18 - DNA, 20 bp, Homo sapiens
caccgcttctgtgaactcct

SEQ ID NO: 19 - DNA, 20 bp, Homo sapiens
tgacatccgaggtgcctttc

SEQ ID NO: 20 - DNA, 20 bp, Homo sapiens
gttagcaactacgcgcaacc

SEQ ID NO: 21 - DNA, 20 bp, Homo sapiens
gtacacctgtccccctgctt

SEQ ID NO: 22 - DNA, 19 bp, Homo sapiens
tgaagcgcactgtcttgct

SEQ ID NO: 23 - DNA, 21 bp, Homo sapiens
gcgagttctacatgatcggct

SEQ ID NO: 24 - DNA, 20 bp, Homo sapiens
ccgtgatcatgtgacgatgc

SEQ ID NO: 25 - DNA, 20 bp, Homo sapiens
atcgttcaggaacacccgac

SEQ ID NO: 26 - DNA, 20 bp, Homo sapiens
cctgttgctggtactgttgg

SEQ ID NO: 27 - DNA, 20 bp, Homo sapiens
agaagccctcaaaacccctg

SEQ ID NO: 28 - DNA, 20 bp, Homo sapiens
cgttgattcgaccacttggc

SEQ ID NO: 29 - DNA, 21 bp, Homo sapiens
tgctgaggatttggaaagggt

SEQ ID NO: 30 - DNA, 20 bp, Homo sapiens
tgatggcctcccatctcctt

SEQ ID NO: 31 - DNA, 20 bp, Homo sapiens
acaacagcctgccaccttac

SEQ ID NO: 32 - DNA, 22 bp, Homo sapiens
tgccataaggcatcattggact

SEQ ID NO: 33 - DNA, 21 bp, Homo sapiens
cctggcattgccgacaggatg

SEQ ID NO: 34 - DNA, 23 bp, Homo sapiens
ccgatccacacggagtacttgcg